(19) United States
(12) Patent Application Publication
(10) Pub. No.: US 2009/0143240 A1
(43) Pub. Date: Jun. 4, 2009

(54) Novel Methods for Genome-Wide Location Analysis

(76) Inventors: Christopher T. Harbison, Hamilton, NJ (US); Richard A. Young, Weston, MA (US); Dmitry K. Pokholok, San Diego, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/064,594

(22) PCT Filed: Aug. 25, 2006
(86) PCT No.: PCT/US06/33325

§ 371 Date: Jul. 29, 2008

Related U.S. Application Data
(60) Provisional application No. 60/711,253, filed on Aug. 25, 2005.

(51) Int. Cl. C40B 30/04; G01N 33/566

(52) U.S. Cl. 506/9; 436/501

(57) ABSTRACT

The invention relates to improved methods of identifying the genomic regions to which a protein of interest binds, and in particular, to methods that use tiled arrays. The invention also provides methods of identifying the transcriptional rate of the gene in a cell. The invention also relates to methods of performing genome-wide location analysis, and ChIP-CHIP analysis, using histones and modified histones.

### CROSS-REFERENCE TO RELATED APPLICATIONS

This application claims the benefit of the filing date of U.S. Application No. 60/711,253, filed Aug. 25, 2005, entitled “NOVEL METHODS FOR GENOME-WIDE LOCATION ANALYSIS.”The entire teachings of the referenced application is incorporated by reference.

### STATEMENT REGARDING FEDERALLY-SPONSORED RESEARCH OR DEVELOPMENT

The invention described herein was supported, in whole or in part, by Grant No. NHGRI grant HG002668 and NIH grant GM069676. The United States govenunent has certain rights in the invention.

### BACKGROUND OF THE INVENTION

Genome-wide analysis methods have been used to determine how tagged transcriptional regulators encoded in Saccharomyces cerevisae are associated with the genome in living yeast cells and to model the transcriptional regulatory circuitry of these cells. These methods have also been used in human tissue culture cells to identify target genes for several transcriptional regulators. Most of these efforts however, provide low-resolution data and relate to unmodified proteins. A need remains, therefore, for developing methods that allow the identification of binding sites on the genome at higher resolutions and that allow the identification of changes in the DNA-binding properties of proteins in response to post-translational modifications. The present invention provides these and other methods.

### SUMMARY OF THE INVENTION

One aspect of the invention provides a method of identifying regions of a genome to which a DNA-binding protein binds, comprising the steps of: (i) obtaining a population of DNA fragments enriched for fragments bound by a DNA-binding protein of interest; (ii) identifying a location on a chromosome to which the DNA-binding protein of interest binds by obtaining results from an array hybridization experiment in which DNA fragment sequences, which have been bound by the protein of interest, are hybridized to a nucleic acid array comprising known sequences, thereby identifying regions of the genome to which the DNA-binding protein binds. In one embodiment, the array is a tiled array.

One aspect of the invention provides a method of identifying a location on a chromosome to which the DNA-binding protein of interest binds, comprising the steps of: (i) crosslinking a DNA-binding protein of interest to a nucleic acid population; (ii) fragmenting nucleic acid molecules bound to the DNA-binding protein of interest; (iii) obtaining a population of DNA fragments bound to the DNA-binding protein of interest; and (iv) identifying a location on a chromosome to which the DNA-binding protein of interest binds by obtaining results from an array hybridization experiment in which DNA fragment sequences, which have been bound to the protein of interest, are hybridized to a nucleic acid array comprising known sequences, thereby identifying regions of a genome to which a DNA-binding protein binds. In one embodiment, the array is a tiled array.

Still another aspect of the invention provides a method of identifying a location on a chromosome to which the DNA-binding protein of interest binds, comprising the steps of: (i) obtaining a population of DNA fragments enriched for fragments which have been bound by a DNA-binding protein of interest; and (ii) identifying a location on a chromosome to which the DNA-binding protein of interest binds by obtaining results from an array hybridization experiment in which DNA fragment sequences, which have been bound by the protein of interest, are hybridized to a nucleic acid array comprising known sequences. In one embodiment, the array is a tiled array.

A further aspect of the invention provides a method of displaying results of an array hybridization experiment, comprising the steps of: (i) obtaining a population of DNA fragments enriched for fragments bound by a DNA-binding protein of interest; and (ii) displaying results of an array hybridization experiment in which DNA fragments bound by the protein of interest are hybridized to a nucleic acid array comprising known sequences. In one embodiment, the array is a tiled array. In certain aspects, the chromosome position of a binding site of the DNA-binding protein of interest is displayed. In certain aspects, the sequence of a binding site of the DNA-binding protein of interest is displayed.

The invention further provides a method, comprising the steps of: (i) comparing the DNA-binding status of a DNA-binding protein of interest at locations of the genome to transcription at the locations of the genome by obtaining results from an array hybridization experiment in which DNA fragment sequences which have been bound by the DNA-binding protein of interest are hybridized to a nucleic acid array; and (ii) comparing the results to gene expression results for the locations. In one embodiment, the array is a tiled array.

The invention also provides a method of identifying a location on a chromosome to which the DNA-binding protein of interest binds, comprising the steps of: (i) obtaining a population of DNA fragments enriched for fragments bound by a DNA-binding protein of interest; (ii) amplifying the enriched fragments; (iii) labeling the enriched DNA fragment sequences; and (iv) providing data from signals obtained from labeled fragment sequences bound to a nucleic acid array comprising know sequences, wherein the data identifies a location on a chromosome to which the DNA-binding protein of interest binds. In one embodiment, the array is a tiled array.

One aspect of the invention provides methods of estimating, determining, or quantitating the transcriptional rate of a gene. Another aspect provides methods of determining the transcriptional rate of a plurality of genes from a genome. The plurality may include, for example, all the genes in a chromosome or portion thereof, or all or most of the genes from a genome or from a fragment thereof. The methods may be used to determine the level of transcription from all or some of the promoters or transcriptional start sites in a genome, and in particular polymerase III promoters in a genome. In one embodiment, the methods of estimating, determining, or quantitating the transcription level of one or more genes in the genome comprises determining the level of at least one histone, or modified histone, in the transcriptional start site of the gene and/or along the coding region of the gene. Table II list preferred histones and modified histones that may be used in the methods of the invention.

### BRIEF DESCRIPTION OF THE DRAWINGS

FIGS. 1A-1D show nucleosome occupancy across the yeast genome with high resolution genome-wide location analysis. (A). Occupancy of the HIS1 promoter by Gcn4. The genomic positions of probe regions are arrayed along the x-axis with the ratio of enrichment of Gcn4 for probes along the y-axis. ORFs are depicted as gray rectangles, and arrows indicate the direction of transcription. Red boxes represent sequence matches to the Gcn4 binding specificity within promoter regions. (B). Composite profile of Gcn4 binding at the set of 84 high-confidence Gcn4 target genes. Promoter and downstream regions were aligned with each other according to the position of a sequence match to the Gcn4 binding specificity. Aligned probes were then assigned to 50-bp segment bins, and an average of the corresponding enrichment ratio was calculated. Standard error of the mean is shown in gray. Genetic elements are depicted as in FIG. 1A, except that dashed lines represent sites including both ORFs and intergenic regions. (C). Nucleosome occupancy at the promoter of CPA1, a gene encoding an amino acid biosynthetic enzyme. The genomic positions of probe regions are arrayed along the x-axis with the ratio of enrichment of histone H3 for probes along the y-axis. ORFs are depicted as gray rectangles, and arrows indicate the direction of transcription. (D). A composite profile of histone occupancy at 5,324 genes. The ends of ORFs were defined at fix points according to the position of translational start and stop sites. The length of the ORF was then subdivided into forty regions of equal length, and probes were assigned according to their nearest corresponding relative position. Probes in promoter regions were similarly assigned following subdivision into twenty regions. The average histone H3 (blue) or H4 (red) enrichment for each subdivided bin is plotted.

FIGS. 2A-2D show comparisons of histone profiles. (A) A composite profile of enrichment in a control experiment. The profile is created as in FIG. 1D, except that enrichment is measured from a mock immunoprecipitation, in which no antibody has been included (Experimental Procedures). (B) A composite profile of histone occupancy normalized to a control. The profile is created as in FIG. 1D, except that enrichment from H3 immunoprecipitation is normalized to enrichment from mock immunoprecipitations. (C) A composite profile of histone occupancy according to transcriptional activity. All genes for which data were available (Holstege et al. 1998) were divided into five classes according to their transcriptional rate. Composite data were computed for H3 enrichment as in FIG. 1D. (D) A composite profile of normalized histone occupancy according to transcriptional activity. The composite profile is created as in FIG. 2C, except that enrichment from H3 immunoprecipitation is normalized to enrichment from mock immunoprecipitations.

FIGS. 3A-3F show nucleosome acetylation generally correlates with transcriptional activity. (A) Acetylation of H3K9 at a locus on chromosome XII. Enrichment is depicted as in FIG. 1. The number beneath each gene represents the transcriptional frequency of the corresponding ORF (Holstege et al., 1998) in mRNA/hr. (B) Composite profile of acetylation of H3K9 across the average gene. Composite profiles of acetylation according to transcriptional frequency class are shown as in FIG. 2. (C) Acetylation of H3K14 at a locus on chromosome XII. Enrichment is depicted as in FIG. 1. The number beneath each gene represents the transcriptional frequency of the corresponding ORF (Holstege et al., 1998) in mRNA/hr. (D) Composite profile of acetylation of H3K14 across the average gene. Composite profiles of acetylation according to transcriptional frequency class are shown as in FIG. 2. (E) Hyperacetylation of H4 at a locus on chromosome XII. Enrichment is depicted as in FIG. 1. The number beneath each gene represents the transcriptional frequency of the corresponding ORF (Holstege et al., 1998) in mRNA/hr. (F) Composite profile of hyperacetylation of H4 across the average gene. Composite profiles of acetylation according to transcriptional frequency class are shown as in FIG. 2.

FIGS. 4A-4F show nucleosome methylation generally correlates with transcriptional activity. (A) Trimethylation of H3K4 at a locus on chromosome XII. Enrichment is depicted as in FIG. 1. The number beneath each gene represents the transcriptional frequency of the corresponding ORF (Holstege et al., 1998) in mRNA/hr. (B) Composite profile of trimethylation of H3K4 across the average gene. Composite profiles of methylation according to transcriptional frequency class are shown as in FIG. 2. (C) Trimethylation of H3K36 at a locus on chromosome XII. Enrichment is depicted as in FIG. 1. The number beneath each gene represents the transcriptional frequency of the corresponding ORF (Holstege et al., 1998) in mRNA/hr. (D) Composite profile of trimethylation of H3K36 across the average gene. Composite profiles of methylation according to transcriptional frequency class are shown as in FIG. 2. (E) Trimethylation of H3K79 at a locus on chromosome XII. Enrichment is depicted as in FIG. 1. The number beneath each gene represents the transcriptional frequency of the corresponding ORF (Holstege et al., 1998) in mRNA/hr. (F) Composite profile of trimethylation of H3K79 across the average gene. Composite profiles of methylation according to transcriptional frequency class are shown as in FIG. 2.

FIG. 5 shows a high-resolution genome-wide map of nucleosome states. A map for a region on chromosome IV is depicted as in FIG. 1. Conserved binding sites for transcriptional regulators (Harbison et al., 2004) are depicted as colored boxes. Numbers beneath genes represent transcriptional activity (mRNA/hr). Enrichment values from acetylated H3K14, trimethylated H3K4 and histone H3 are depicted in red, green and black, respectively.

FIGS. 6A-6F show positive and negative examples of Gcn4 binding. (A) Occupancy of the ARG3 promoter by Gcn4. The genomic positions of probe regions are arrayed along the x-axis with the ratio of enrichment of Gcn4 for probes along the y-axis. ORFs are depicted as gray rectangles, and arrows indicate the direction of transcription. Red boxes represent sequence matches to the Gcn4 binding specificity within promoter regions. (B) Occupancy of the ARO1 promoter by Gcn4 as in A. (C) Occupancy of the CPA1 promoter by Gcn4 as in A. D. Occupancy of the MET13 promoter by Gcn4 as in A. (E) Occupancy of the LEU4/MET4 promoter by Gcn4 as in A. (F) Occupancy of the PDC5 and SLX4 promoter by Gcn4 as in A (negative control)

FIG. 7 shows histone H3 occupancy at the CPA1 locus after normalization to a no-antibody control.

FIGS. 8A-8D show changes in nucleosome occupancy in response to changes in environmental conditions. (A) Changes in nucleosome occupancy at the HSP30 locus in response to hyperoxia (red), YPD (blue) (B) Changes in nucleosome occupancy at the HSP82 locus in response to hyperoxia (red), YPD (blue) (C) A sliding window (size=100) of H3 enrichment as a function of hyperoxia-induced changes in gene expression as determined in (Causton et al., 2001), hyperoxia (red), YPD (blue) (D) A composite profile of H3 occupancy for sets of genes according to changes in transcriptional activity, genes induced more than 10 fold in bright red, induced 10 to 2 fold—dark red, induced up to 2 fold—gold, repressed up to 2 fold—dark green, repressed more than 2 fold—bright green.

FIGS. 9A-9B show correlation of Histones H3 and H4 acetylation with Gcn5 and Esa1 occupancy. (A) A sliding window of Gcn5 occupancy (size=100) was compared to histone H3 acetylation at lysine 14 (H3K14ac) (B) A sliding window of Esa1 occupancy (size=100) was compared to histone H4 acetylation (H4ac).

FIGS. 10A-10D show changes in acetylation of Histone H3 lysine 14 (H3K14ac) in response to changes in environmental conditions. (A) Changes in nucleosome acetylation at the HSP30 locus in response to hyperoxia (red), YPD (blue) (B) Changes in nucleosome acetylation at the HSP82 locus in response to hyperoxia (red), YPD (blue) (C) A sliding window (size=100) of H3 acetylation as a function of hyperoxia-induced changes in gene expression as determined in (Causton et al., 2001), hyperoxia (red), YPD (blue) (D) A composite profile of nucleosome acetylation for sets of genes according to changes in transcriptional activity, genes induced more than 10 fold in bright red, induced 10 to 2 fold—dark red, induced up to 2 fold—gold, repressed up to 2 fold—dark green, repressed more than 2 fold—bright green. Similar, though less pronounced, effects were observed for H4ac.

FIGS. 11A-11B show correlation of Histone H3 acetylation at lysine 14 (H3K14ac) with transcriptional activity. (A) A sliding window of H3 acetylation (size=100) within ORFs compared to transcriptional activity in mRNA/hr (Holstege et al. 1998) Red line—H3K14ac vs. WCE, blue line—H3K14ac vs. H3. (B) A sliding window of H3 acetylation (size=100) within intergenic regions compared to the transcriptional activity of downstream genes in mRNA/hr (Holstege et al. 1998) Red line—H3K14ac vs. WCE, blue line—H3K14ac vs. H3.

FIGS. 12A-12D show differential profiles of methylated H3K4. (A) Profiles of mono-(blue), di-(red) and tri-methylated (grey) H3K4 are shown at a portion of Chromosome XII. (B) Composite profiles of mono-(blue), di-(red) and trimethylated (grey) H3K4 at the average gene. (C) Composite profiles of monomethylated H3K4 according to transcriptional activity. (D) Composite profiles of dimethylated H3K4 according to transcriptional activity.

FIG. 13 show distributions of median H3 enrichment at ORF and intergenic regions before and after normalization to control experiments. Boxes represent the 10th to the 90th percentiles, vertical lines extend to the 5th and 95th percentiles, and horizontal bars represent the mean of the entire sample. We used a two-sampled T-test to determine the likelihood of obtaining different values for the mean enrichment by chance in both the original and controlled experiments. Assuming unequal variance between the two regions, we found a likelihood estimate of <10⁻¹⁶ using non-normalized H3 data. Corrected using the IgG data, this value increases to 0.45, suggesting the differences in between the two regions are insignificant.

FIGS. 14A-14D show oligonucleotide features along a region of a genome containing an exon. FIG. 14A shows exon 1400 and fully overlapping oligonucleotide features 1401. FIG. 14B shows exon 1410 and partially overlapping oligonucleotide features 1411. FIG. 14C shows exon 1420 and spaced oligonucleotide features 1421. FIG. 14D shows exon 1430 and non-overlapping adjacent oligonucleotide features 1431.

### DETAILED DESCRIPTION OF THE INVENTION

### I. Overview

The invention provides, in part, methods of identifying regions of a genome to which a protein of interest binds in a cell. One aspect of the invention provides a method of identifying regions of a genome to which a protein of interest binds comprising the steps of: (a) producing a mixture comprising DNA fragments to which the protein of interest is bound; (b) isolating one or more DNA fragments to which the protein of interest is bound from the mixture produced in step (a); and (c) identifying regions of the genome which are complementary to the DNA fragments isolated in step (b), thereby identifying regions of the genome to which the protein of interest binds. In one embodiment, the method comprises, between steps (b) and (c), the step of generating a probe from the one or more of the isolated DNA fragments. In another embodiment, the probe is labeled with a fluorescent probe. In one embodiment, step (c) comprises combining the probe with one or more sets of distinct oligonucleotide features bound to a surface of a solid support, wherein the distinct oligonucleotide features are each complementary to a region of the genome, under conditions in which specific hybridization between the probe and the oligonucleotide features can occur, and detecting said hybridization, wherein hybridization between the labeled probe and a oligonucleotide feature relative to a suitable control indicates that the protein of interest binds to the region of the genome to which the sequence of the oligonucleotide feature is complementary.

One aspect of the invention provides a method for identifying regions of a genome to which a protein of interest binds, the method comprising the steps of: (a) producing a mixture comprising DNA fragments to which the protein of interest is bound; (b) isolating one or more DNA fragments to which the protein of interest is bound from the mixture produced in step (a); (c) generating probes from the one or more of the isolated DNA fragments; and (d) identifying one or more regions of the genome which are complementary to the probe fragments isolated in step (c) by combining the probe with a tiled array comprising one or more sets of distinct oligonucleotide features bound to a surface of a solid support, wherein the distinct oligonucleotide features are each complementary to a region of the genome, thereby identifying regions of the genome to which the protein of interest binds. In one embodiment, step (d) comprises combining the probe and the tiled array under conditions in which specific hybridization between the probe and the oligonucleotide features can occur, and detecting said hybridization, wherein hybridization between the labeled probe and a oligonucleotide feature relative to a suitable control indicates that the protein of interest is bound to the region of the genome to which the sequence of the oligonucleotide feature is complementary.

In another embodiment, one or more sets of the distinct oligonucleotide features are complementary to locations in the genome that are substantially evenly spaced. In another embodiment, the distinct oligonucleotide features are complementary to adjacent regions in the genome that are spaced from 10 bp to 20 kb, or from 20 bp to 10 kb, of each other. In one embodiment, the oligonucleotide features comprise DNA or RNA or modified forms thereof. In one embodiment, the oligonucleotide features are DNA oligonucleotides, or RNA oligonucleotides, which may be optionally modified so that one or more nucleotides is a non-naturally ocurring nucleotide. In one embodiment, the modified forms of DNA are PNA or LNA molecules. In one embodiment, wherein said oligonucleotide features comprise nucleic acids that range in size from about 20 nucleotides (nt) to about 200 nt in length. In one embodiment, the nucleic acids range in size from about 20 to about 100 nt in length. In one embodiment, the nucleic acids range in size from about 40 to about 80 nt in length.

In one embodiment, the oligonucleotide features bound to a surface of a solid support include sequences representative of locations distributed across at least a portion of a genome. In one embodiment, the locations have a uniform spacing across at least a portion of a genome. In another embodiment, the locations have a non-uniform spacing across at least a portion of a genome. In another embodiment, the one or more sets of oligonucleotide features bound to a surface of a solid support samples the portion of the genome at least about every 1, 2, 3, 4, 5, 7, 10, 12, 14, 16 18 or 20 Kb. In another embodiment, the one or more sets of oligonucleotide features bound to a surface of a solid support samples at least a portion of the genome at least about every 2 Kb. In yet another embodiment, the one or more sets of oligonucleotide features bound to a surface of a solid support samples at least a portion of the genome at least about every 0.5 Kb.

In yet one embodiment, the portion of the genome comprises at least 20%, 30%, 40%, 50%, 60%, 70%, 80%, 90%, and 95% of the genome. In one embodiment, the portion of the genome comprises regions of at least 20%, 30%, 40%, 50%, 60%, 70%, 80%, 90%, and 95% of the chromosomes in the genome. In another embodiment, at least one, two, three or more sets of distinct oligonucleotide features comprise distinct oligonucleotide features that correspond to non-coding genomic regions. In another embodiment, at least 50%, 60%, 70%, 80%, 90% or 95% of said sets of distinct oligonucleotide features are complementary to non-promoter regions.

In one embodiment, at least one set of distinct oligonucleotide features comprises distinct oligonucleotide features that correspond to coding genomic regions. In another embodiment, at least 50%, 60%, 70%, 80%, 90% or 95% of the distinct oligonucleotide features that correspond to coding genomic regions do not comprise entire or partial open reading frames. In one embodiment, the solid support is a planar substrate such as glass. In one embodiment, the sets of distinct oligonucleotide features bound to a solid surface comprise an array, such as a tiled array.

In one embodiment, steps (a) and (b) are performed in a first location, and step (c) is performed in a second location, wherein the first location is remote to the second location. In one embodiment, the DNA fragments to which the protein of interest is bound from the mixture produced in step (a), or the labeled probes derived from said DNA fragments, are delivered from the first location to the second location. In another embodiment, the method comprises a data transmission step between the first location and the second location. In one embodiment, the data transmission step occurs via an electronic communication link such as the internet.

In one embodiment, the data transmission step from the first to the second location comprises experimental parameter data, wherein the experimental parameter data comprises data selected from: (a) the phylogenetic species of the genome; (b) clinical data from the organism from which the genome was derived; and (c) a microarray to which the labeled probes are to be hybridized. In another embodiment, the data transmission step from the second location to the first location comprises (i) one or more data transmission substeps from the second location to one or more intermediate location; and (b) one or more data transmission substeps from one or more intermediate location to the first location, wherein the intermediate locations are remote to both the first and second locations.

In one embodiment, the method further comprises a data transmission step in which a result from identifying regions of a genome is transmitted from the second location to the first location. In another embodiment, the data transmission step from the second location to the first location comprises (i) one or more data transmission substeps from the second location to one or more intermediate location; and (b) one or more data transmission substeps from one or more intermediate location to the first location, wherein the intermediate locations are remote to both the first and second locations. In one embodiment, the data transmission step occurs via the an electronic communication link. In one specific embodiment, the data communication link is the internet.

In one embodiment, the genome is from an eukaryotic cell. In another embodiment, the cell is a metazoan cell. In another embodiment, the cell is a mammalian cell. In one embodiment, the cell is a primary cell. In one embodiment, the cell is derived or isolate from a tissue biopsy, preferably without in vitro expansion. In one embodiment, the tissue biopsy is from a subject afflicted with, or suspected of being afflicted with, a disorder such as cancer, a viral infection, an autoimmune disease or a neurodegenerative disease. In one preferred embodiment, the cell is a human cell. In one embodiment, the cell is a non-human cell, such as a mammalian non-human cell. In another embodiment, the cell is a yeast cell.

In one embodiment, the protein of interest is a sequence-specific DNA-binding protein. In another embodiment, the protein of interest is not a sequence-specific DNA-binding protein. In one embodiment, the protein of interest is acetylated, methylated, or both. In one embodiment, the protein of interest is native to the cell. In one embodiment, the protein of interest is a recombinant protein. In one embodiment, the protein of interest is a histone. In a specific embodiment, the histone is an unmodified histone. In one embodiment, the histone is a modified histone. In one embodiment, the histone is acetylated, methylated, phosphorylated, or combinations thereof. In one embodiment, the histone is selected from H3, H4, H3K9ac, H3K14ac, H4K5acK8acK12acK16ac, H3K4me, H3K4me2, H3K4me3, H3K36me3 and H3K79me3. In one embodiment, the histone is selected from H3K9ac, H3K14ac, H4K5acK8acK12acK16ac, H3K4me, H3K4me2, H3K4me3, H3K36me3 and H3K79me3. In one embodiment, the histone is selected from H3K9ac, H3K14ac, and H4K5acK8acK12acK16ac. In one embodiment, the histone is selected from H3K4me, H3K4me2, H3K4me3, H3K36me3 and H3K79me3.

In one embodiment, the genome is from a first cell and the protein of interest from a second cell. In one embodiment, the method comprises the step, prior to step (a), of contacting the protein of interest with the genome. In one embodiment, the protein of interest is contacted with the genome ex vivo by contacting (i) an extract comprising the protein; and (ii) an extract comprising the genome. In one embodiment, the protein of interest is a recombinant protein. In one embodiment, the protein of interest is a naturally-occurring protein. In one embodiment, the first cell and the second cells are from different species.

One aspect of the invention provides a method of identifying a gradient of binding of a protein of interest within a plurality of genes in the genome of a cell, the method comprising (i) identifying regions of the genome of the cell to which a protein of interest is bound; and (ii) comparing the frequency of binding of the protein of interest along the coding region of the plurality of genes, wherein the oligonucleotide features bound to a surface of a solid support include oligonucleotide features complementary to multiple regions within the coding regions of the plurality of genes. In one embodiment, the plurality of genes includes at least 50% of the genes in the genome. In one embodiment, the method further comprises normalizing the length of the coding regions of the plurality of genes to identify a normalized aggregate gradient of binding along the coding regions of the genes. In one embodiment, the multiple regions within the coding regions comprise, on average, at least 2 regions. In another embodiment, the protein of interest is not a sequence-specific DNA-binding protein. In one embodiment, the cell has been contacted with a drug.

One aspect of the invention provides a method of estimating the transcriptional rate of a gene, the method comprising determining the level of acetylated histone bound to a transcriptional start site of the gene, wherein increased levels of bound acetylated histone indicate a higher transcriptional rate. In one embodiment, the transcriptional rate is relative transcriptional rate, such as relative to a reference gene. In one aspect, the acetylated histone in monoacetylated. In another embodiment, the acetylated histone is multiply acetylated. In another embodiment, determining the level of acetylated histone bound to a transcriptional start site of the gene comprises determining the regions of the genome to which the acetylated histone binds using genome-wide location analysis or CHIP-CHIP analysis. In one embodiment, the acetylated histone is H3 acetylated at K9, H3 acetylated at K14, or H4 acetylated at K5, K8, K12 and K16.

One aspect of the invention provides a method of estimating the transcriptional rate of a gene, the method comprising determining the level of methylated histone bound to the transcribed region the gene, the coding region of the gene, or the open reading frame of the gene, wherein increased levels of methylated histone bound to the transcribed region indicate a higher transcriptional rate. In one embodiment, the transcriptional rate is relative transcriptional rate, such as relative to a reference gene. In one embodiment, the methylated histone is trimethylated. In one embodiment, the methylated histone is H3 methylated at K36. In one embodiment, the methylated histone is H3 trimethylated at K36. In one embodiment, determining the relative level of methylated histone bound to the transcribed region of a gene comprises determining the regions of the genome to which the methylated histone binds using the methods provided for identifying region(s) on a chromosome to which a protein of interest binds. In one embodiment, the transcribed region or the gene is the coding sequence. In one embodiment, the method comprises determining the level of methylated histone bound to the 3′ region of the transcribed portion of the gene, and the histone is preferable K36 trimethylated histone. In one embodiment, the 3′ region refers to a portion of the gene corresponding to approximately 50%, 45%, 40%, 35%, 30%, 25%, 20%, 15%, 10%, 5% or less of the 3′ end of the transcribed portion of the gene.

Another aspect of the invention comprises identifying a plurality of ORF in a genome, the method comprising identifying the regions of a genome to which a modified histone binds, wherein the modified histone is H3 with dimethylation or monomethylation at K4, or with trimethylation at K36 or K79.

In one embodiment, all the methods described herein are carried ex-vivo and do not involve any manipulation or a human or mammalian body.

### II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to.”

A “patient” or “subject” to be treated by the method of the invention can mean either a human or non-human animal, preferably a mammal.

The term “encoding” comprises an RNA product resulting from transcription of a DNA molecule, a protein resulting from the translation of an RNA molecule, or a protein resulting from the transcription of a DNA molecule and the subsequent translation of the RNA product.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

“Recombinant” when used with-reference, e.g., to a nucleic acid, cell, virus, plasmid, vector, or the like, indicates that these have been modified by the introduction of an exogenous, non-native nucleic acid or the alteration of a native nucleic acid, or have been derived from a recombinant nucleic acid, cell, virus, plasmid, or vector. Recombinant protein refers to a protein derived from a recombinant nucleic acid, virus, plasmid, vector, or the like.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “microarray” refers to an array of distinct polynucleotides or oligonucleotides synthesized on a substrate, such as paper, nylon or other type of membrane, filter, chip, glass slide, or any other suitable solid support.

A probe that is “labeled” is detectable, either directly or indirectly, by spectroscopic, photochemical, biochemical, immunochemical, isotopic, or chemical means. For example, useful labels include ³²P, ³³P, ³⁵S, ¹⁴C, ³H, ¹²⁵I, stable isotopes, fluorescent dyes and fluorettes (Rozinov and Nolan (1998) Chem. Biol 5:713-728; Molecular Probes, Inc. (2003) Catalogue, Molecular Probes, Eugene Oreg.), electron-dense reagents, enzymes and/or substrates, e.g., as used in enzyme-linked immunoassays as with those using alkaline phosphatase or horse radish peroxidase. The label or detectable moiety is typically bound, either covalently, through a linker or chemical bound, or through ionic, van der Waals or hydrogen bonds to the molecule to be detected. “Radiolabeled” refers to a compound to which a radioisotope has been attached through covalent or non-covalent means. A “fluorophore” is a compound or moiety that absorbs radiant energy of one wavelength and emits radiant energy of a second, longer wavelength.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe can be detected by detecting the presence of the label bound to the probe. The probes are preferably directly labeled as with isotopes, chromophores, fluorophores, chromogens, or indirectly labeled such as with biotin to which a streptavidin complex or avidin complex can later bind.

A “nucleic acid probe” is a nucleic acid capable of binding to a target nucleic acid of complementary sequence, usually through complementary base pairing, e.g., through hydrogen bond formation. A probe may include natural, e.g., A, G, C, or T, or modified bases, e.g., 7-deazaguanosine, inosine, etc. The bases in a probe can be joined by a linkage other than a phosphodiester bond. Probes can be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions.

“Polymerase chain reaction” (PCR) refers, e.g., to a procedure or product where a specific region or segment of a nucleic acid is amplified, and where the segment is bracketed by primers used by DNA polymerase (Bernard and Wittwer (2002). Clin. Chem. 48: 1178-1185; Joyce (2002) Methods Mol. Biol. 193:83-92; Ong and Irvine (2002) Hematol. 7:59-67).

A “promoter” is a nucleic acid sequence that directs transcription of a nucleic acid. A promoter includes nucleic acid sequences near the start site of transcription, e.g., a TATA box, see, e.g., Butler and Kadonaga (2002) Genes Dev. 16:2583-2592; Georgel (2002) Biochem. Cell Biol. 80:295-300. A promoter also optionally includes distal enhancer or repressor elements, which can be located as much as several thousand base pairs on either side from the start site of transcription. A “constitutive” promoter is a promoter that is active under most environmental and developmental conditions, while an “inducible” promoter is a promoter is active or activated under, e.g., specific environmental or developmental conditions.

“Small molecule” is defined as a molecule with a molecular weight that is less than 10 kD, typically less than 2 kD, and preferably less than 1 kDa. Small molecules include, but are not limited to, inorganic molecules, organic molecules, organic molecules containing an inorganic component, molecules comprising a radioactive atom, synthetic molecules, peptide mimetics; and antibody mimetics. As a therapeutic, a small molecule may be more permeable to cells, less susceptible to degradation, and less apt to elicit an immune response than large molecules. Small molecule toxins are described, see, e.g., U.S. Pat. No. 6,326,482 issued to Stewart, et al.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism. For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other nucleic acids that are C-glycosides of a purine or pyrimidine base, polypeptides (proteins) or polysaccharides (starches, or polysugars), as well as other chemical entities that contain repeating units of like chemical structure.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. The terms “ribonucleic acid” and “RNA” as used herein mean a polymer composed of ribonucleotides. The terms “deoxyribonucleic acid” and “DNA” as used herein mean a polymer composed of deoxyribonucleotides. The term “oligonucleotide” as used herein denotes single stranded nucleotide multimers of from about 10 to 100 nucleotides and up to 200 nucleotides in length.

The term “functionalization” as used herein relates to modification of a solid substrate to provide a plurality of functional groups on the substrate surface. By a “functionalized surface” is meant a substrate surface that has been modified so that a plurality of functional groups are present thereon.

The terms “reactive site”, “reactive functional group” or “reactive group” refer to moieties on a monomer, polymer or substrate surface that may be used as the starting point in a synthetic organic process. This is contrasted to “inert” hydrophilic groups that could also be present on a substrate surface, e.g., hydrophilic sites associated with polyethylene glycol, a polyamide or the like.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO.sub.4, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

Sensitivity is a term used to refer to the ability of a given assay to detect a given analyte in a sample, e.g., a nucleic acid species of interest. For example, an assay has high sensitivity if it can detect a small concentration of analyte molecules in sample. Conversely, a given assay has low sensitivity if it only detects a large concentration of analyte molecules (i.e., specific solution phase nucleic acids of interest) in sample. A given assay's sensitivity is dependent on a number of parameters, including specificity of the reagents employed (e.g., types of labels, types of binding molecules, etc.), assay conditions employed, detection protocols employed, and the like. In the context of array hybridization assays, such as those of the present invention, sensitivity of a given assay may be dependent upon one or more of: the nature of the surface immobilized nucleic acids, the nature of the hybridization and wash conditions, the nature of the labeling system, the nature of the detection system, etc.

### III. Methods of Identifying Chromosome Regions

One aspect of the invention provides methods for identifying a region of a genome of a cell to which a protein of interest binds. One aspect provides a method of identifying the regions of nuclear DNA to which a DNA-binding protein is bound in a cell. One specific aspect of the invention provides a method for identifying at least one region of a genome to which a protein of interest binds, the method comprising the steps of: producing a mixture comprising DNA fragments to which the protein of interest is bound; (b) isolating one or more DNA fragments to which the protein of interest is bound from the mixture produced in step (a); and (c) identifying regions of the genome which are complementary to the DNA fragments isolated in step (b), thereby identifying at least one (one or more) region of the genome to which the protein of interest is bound. In some embodiments, the method further comprises generating a probe from the one or more of the isolated DNA fragments, such as between steps (b) and (c). In one embodiment, the probe comprises a nucleic acid, which may comprise a detectable label.

In one embodiment of the methods described herein, the protein of interest is covalently crosslinked to the genomic DNA prior to fragmenting the genomic DNA. There are a variety of methods which can be used to link a DNA-binding to genomic DNA. In one embodiment of the methods described herein, the crosslinking is formaldehyde crosslinking (Solomon, M. J. and Varshavsky, A., Proc. Natl. Sci. USA 82:6470-6474; Orlando, V., TIBS, 25:99-104). UV light may also be used (Pashev et al. Trends Biochem Sci. 1991; 16(9):323-6; Zhang L et al. Biochem Biophys Res Commun. 2004; 322(3):705-11).

In one embodiment of the methods described herein where the protein of interest is covalently crosslinked to the genomic DNA prior to fragmenting the genomic DNA of the cell, separating the DNA fragment from the protein of interest comprises the step of reversing the crosslink. In a specific embodiment, it comprises the steps of (i) isolating a DNA fragment to which the protein of interest is bound from the mixture produced in (a); and (ii) separating (1) the DNA fragment from (2) the protein of interest. In a specific embodiment, separating the DNA fragment from the protein of interest to which it is bound comprises the steps of removing the crosslink between the DNA fragment and the protein of interest and removing the protein of interest from the DNA fragment. This may be accomplished, for example, by degrading the protein of interest. In one embodiment, a protease such as proteinase K is used to degrade the protein of interest.

Suitable non-limiting methods for purifying the DNA fragment include column chromatography (U.S. Pat. No. 5,707,812), the use of hydroxylated silica polymers (U.S. Pat. No. 5,693,785), rehydrated silica gel (U.S. Pat. No. 4,923,978), boronated silicates (U.S. Pat. No. 5,674,997), modified glass fiber membranes (U.S. Pat. Nos. 5,650,506; 5,438,127), fluorinated adsorbents (U.S. Pat. No. 5,625,054; U.S. Pat. No. 5,438,129), diatomaceous earth (U.S. Pat. No. 5,075,430), dialysis (U.S. Pat. No. 4,921,952), gel polymers (U.S. Pat. No. 5,106,966) and the use of chaotropic compounds with DNA-binding reagents (U.S. Pat. No. 5,234,809). Commercially available DNA isolation and purification kits are also available from several sources including Stratagene (CLEARCUT Miniprep Kit), and Life Technologies (GLASSMAX DNA Isolation Systems).

In some embodiments of the methods described herein, the genomic DNA is fragmented mechanically, such as by hydrodynamic shearing or sonication. Mechanical fragmentation can occur by any method known in the art, including shearing of DNA by passing it through the narrow capillary or orifice (Oefner et al., 1996, Nucleic Acids Res.;24(20):3879-86; Thorstenson et al., 1998, Genome Res.; 8(8):848-55), sonicating the DNA, such as by ultrasound (Bankier, 1993, Methods Mol. Biol.; 23:47-50, or grinding in cell homogenizers (Rodriguez L V. Arch Biochem Biophys. 1980; 200(1): 116-29). Mechanical fragmentation usually results in double strand breaks within the DNA molecule. Sonication may also be performed with a tip sonicator, such as a multi-tip sonicator, or more preferably using acoustic soundwaves. A Microplate Sonicator® (Misonix Inc.) may be used to partially fragment the DNA. Such a device is described in U.S. Patent Publication No. 2002/0068872. Another acoustic-based system that may be used to fragment DNA is described in U.S. Pat. No. 6,719,449, manufactured by Covaris Inc. U.S. Pat. No. 6,235,501 describes a mechanical method of producing high molecular weight DNA fragments by application of rapidly oscillating reciprocal mechanical energy to cells in the presence of a liquid medium in a closed container, which may be used to mechanically fragment the DNA.

Genomic sequences may be amplified prior to or after a fragmentation step. In one embodiment, an amplification step is used which does not substantially reduce the complexity of the initial source of nucleic acids, e.g., genomic DNA is obtained without a pre-selection step or genomic DNA which has been enriched by selecting for fragments which bind to a protein of interest, and amplification employs a random set of primers or primers whose complements occur at a desired frequency throughout the genome or whose complements are engineered to be included in a plurality (e.g., all) genomic fragments obtained from a sample (e.g., such as linkers ligated to the ends of genomic fragments).

However, in other embodiments, amplification can be performed which enriches for certain types of sequences, e.g., sequences which contains a consensus binding site for a protein of interest.

Methods for amplifying nucleic sequences can vary. In one aspect, nucleic acids are amplified using an isothermal amplification technique. In another aspect, nucleic acids are amplified using a strand displacement technique, such as multiple strand displacement. In a further aspect, the nucleic acid is amplified using random primers, degenerate primers and/or primers which bind to a constant sequence ligated to ends of genomic fragments in a sample.

In certain aspects, amplified isolated DNA fragments are labeled, e.g., labeled probes are generated from the fragments by labeling an amplification product of the fragments using methods known in the art.

In a preferred embodiment, the chromatin fragments bound by the protein of interest (e.g. a transcriptional regulator or a histone) are isolated using chromatin immunoprecipitation (ChIP). Briefly, this technique involves the use of a specific antibody to immunoprecipitate chromatin complexes comprising the corresponding antigen i.e. the protein of interest, and examination of the nucleotide sequences present in the immunioprecipitate. Immunoprecipitation of a particular sequence by the antibody is indicative of interaction of the antigen with that sequence. See, for example, O'Neill et al. in Methods in Enzymology, Vol. 274, Academic Press, San Diego, 1999, pp. 189-197; Kuo et al. (1999) Method 19:425-433; and Ausubel et al., supra, Chapter 21. Accordingly, in one embodiment, the DNA fragment bound by the protein of interest is identified using an antibody which binds to the protein of interest.

In one embodiment, the chromatin immunoprecipitation technique is applied as follows in the context of a histone. Cells which express the histone are treated with an agent that crosslinks the histone to chromatin, such as with formaldehyde treatment or ultraviolet irradiation. Subsequent to crosslinking, cellular nucleic acid is isolated, fragmented and incubated in the presence of an antibody directed against the histone. Antibody-antigen complexes are precipitated, crosslinks are reversed (for example, formaldehyde-induced DNA-protein crosslinks can be reversed by heating) so that the sequence content of the immunoprecipitated DNA is tested for the presence of one or more specific sequences. The antibody may bind directly to an epitope on the histone or it may bind to an affinity tag on the histone, such as a myc tag recognized by an anti-Myc antibody (Santa Cruz Biotechnology, sc-764). A non-antibody agent with affinity for the transcriptional regulator, or for a tag fused to it, may be used in place of the antibody. For example, if the histone comprises a six-histidine tag, complexes may be isolated by affinity chromatography to nickel-containing sepharose. Additional variations on CHIP methods may be found in Kurdistani et al. Methods. 2003 31(1):90-5; O'Neill et al. Methods. 2003, 31(1):76-82; Spencer et al., Methods. 2003; 31(1):67-75; and Orlando et al. Methods 11: 205-214 (1997).

In one embodiment of the methods described herein, DNA fragments from a control immunoprecipitation reaction are used in place of the isolated chromatin as a control. For example, an antibody that does not react with a histone being tested may be used in a chromatin IP procedure to isolate control chromatin, which can then be compared to the chromatin isolated using an antibody that binds to the histone. In preferred embodiments, the antibody that does not bind to the histone being tested also does not react with other histone or other DNA-binding proteins. In one embodiment, the suitable control is not whole chromatin DNA. In a preferred embodiment, a suitable control comprises chromatin DNA which had been immunoprecipitated in the presence of a control antibody, such as one that does not bind to the protein of interest, or in the absence of an antibody. In one embodiment, control chromatin is one that has been immunoprecipitated in the presence of an antibody that those not bind to the protein of interest or in the absence of any antibody.

The identification of genomic regions from the isolated DNA fragments may be achieved by generating DNA or RNA probes from the fragment (such as by using the isolated DNA fragments as templates for DNA or RNA synthesis), and hybridizing them to a DNA microarray, such as a DNA microarray comprising immobilized nucleic acids complementary to regions of the genome. In one embodiment, the probes are labeled to facilitate their detection. The probes may be labeled during their synthesis, such as by synthesizing them in the presence of labeled nucleotides, or they may be labeled subsequent to their synthesis. In other embodiments, detection agents may be used to label the DNA/RNA probes once they have hybridized to a DNA microarray. Such detection agents include antibodies, antibody fragments, and dendrimers among others.

In one embodiment, labeled probes are generated by using the DNA fragments as templates for DNA or RNA synthesis by polymerases using techniques well known in the art, such as using the polymerase chain reaction. DNA synthesis may be primed using random primers. Random priming is described in U.S. Pat. Nos. 5,106,727 and 5,043,272. In some embodiments, the labeled probes are generated using ligation-mediated polymerase chain reaction (LM-PCR). LM-PCR is described, for example, in U.S. Publication No. 2003/0143599. Other methods for DNA labeling include direct labeling, 77 RNA polymerase amplification, aminoallyl labeling and hapten-antibody enzymatic labeling. In one embodiment, the labeled probes comprise a fluorescent molecule, such as Cy3 or Cy5 dyes. In another embodiment, the labeled probes comprise semiconducting nanocrystals, also known as quantum dots. Quantum dots are described in U.S. Publication Nos. 2003/0087239 and 2002/0028457, and in international PCT publication No. WO01/61040.

Extension products that are produced as described above are typically labeled in the present methods. As such, the reagents employed in the subject primer extension reactions typically include a labeling reagent, where the labeling reagent may be the primer or a labeled nucleotide, which may be labeled with a directly or indirectly detectable label. A directly detectable label is one that can be directly detected without the use of additional reagents, while an indirectly detectable label is one that is detectable by employing one or more additional reagent, e.g., where the label is a member of a signal producing system made up of two or more components. In many embodiments, the label is a directly detectable label, such as a fluorescent label, where the labeling reagent employed in such embodiments is a fluorescently tagged nucleotide(s), e.g., dCTP. Fluorescent moieties which may be used to tag nucleotides for producing labeled nucleic acids include, but are not limited to: fluorescein, the cyanine dyes, such as Cy3, Cy5, Alexa 555, Bodipy 630/650, and the like. Other labels may also be employed as are known in the art.

When control probes are used, the control probes may be labeled with the same label or different labels as the experimental probes, depending on the actual assay protocol employed. For example, where each set of probes is to be contacted with different but identical arrays, each set of probes may carry the same label. Alternatively, where both sets are to be simultaneously contacted with a single array of immobilized oligonucleotide features, the sets may be differentially labeled.

In some embodiments, the nucleic acid probes are not labeled. For example, in certain embodiments, binding events on the surface of a substrate (such as an oligonucleotide microarray) may be detected by means other than by detection of a labeled nucleic acids, such as by change in conformation of a conformationally labeled immobilized oligonucleotide, detection of electrical signals caused by binding events on the substrate surface, etc.

In one embodiment, identifying a region of the genome of the cell which is complementary to the isolated DNA fragments comprises combining the probe(s) with one or more sets of distinct oligonucleotide features bound to a surface of a solid support under conditions such that nucleic acid hybridization to the surface immobilized features can occur, wherein the distinct oligonucleotide features are each complementary to a region of the genome, under conditions in which specific hybridization between the probe and the oligonucleotide features can occur, and detecting said hybridization, wherein hybridization between the probe and the oligonucleotide features relative to a suitable control indicates that the protein of interest is bound to the region of the genome to which the sequence of the oligonucleotide features is complementary. “Specific hybridization” refers to hybridization occurring under stringent conditions.

The experimental and control probes can be contacted to the surface immobilized features either simultaneously or serially. In many embodiments the compositions are contacted with the plurality of surface immobilized features, e.g., the array of distinct oligonucleotides of different sequence, simultaneously. Depending on how the collections or populations are labeled, the collections or populations may be contacted with the same array or different arrays, where, when the collections or populations are contacted with different arrays, the different arrays are substantially, if not completely, identical to each other in terms of feature content and organization.

An oligonucleotide bound to a surface of a solid support refers to an oligonucleotide or mimetic thereof, e.g., PNA or LNA molecule, that is immobilized on a surface of a solid substrate in a feature or spot, where the substrate can have a variety of configurations, e.g., a sheet, bead, or other structure. In certain embodiments, the collections of features of oligonucleotides employed herein are present on a surface of the same planar support, e.g., in the form of an array.

Arrays refer to an ordered array presented for binding to nucleic acids and the like, and include microarrays. Arrays, as described in greater detail below, are generally made up of a plurality of distinct or different features. The term “feature” is used interchangeably herein with the terms: “features,” “feature elements,” “spots,” “addressable regions,” “regions of different moieties,” “surface or substrate immobilized elements” and “array elements,” where each feature is made up of oligonucleotides bound to a surface of a solid support, also referred to as substrate immobilized nucleic acids. An “array,” includes any one-dimensional, two-dimensional or substantially two-dimensional (as well as a three-dimensional) arrangement of addressable regions (i.e., features, e.g., in the form of spots) bearing nucleic acids, particularly oligonucleotides or synthetic mimetics thereof (i.e., the oligonucleotides defined above), and the like. Where the arrays are arrays of nucleic acids, the nucleic acids may be adsorbed, physisorbed, chemisorbed, or covalently attached to the arrays at any point or points along the nucleic acid chain. Exemplary arrays are described in U.S. Patent Pub No. 2004/0191813.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100μ², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

The number of nucleic acid features of an array may vary, where the number of features present on the surface of the array may be at least 2, 5, or 10 or more such as at least 20 and including at least 50, where the number may be as high as about 100, as about 500, as about 1000, as about 5000, as about 10000 or higher. In representative embodiments, the subject arrays have a density ranging from about 100 to about 100,000 features/cm², such as from about 500 to about 20,000 features/cm², including from about 1000 to about 20,000 features/cm². In representative embodiments, the density of single-stranded nucleic acids within a given feature is selected to optimize efficiency of the RNA polymerase. In certain of these representative embodiments, the density of the single-stranded nucleic acids may range from about 10-3 to about 1 pmol/mm², such as from about 10-2 to about 0.1 pmol/mm², including from about 5×10⁻² to about 0.1 pmol/mm².

In certain aspects, even at high density (e.g., at least about 10,000 features/cm², at least about 50,000 features/cm², or at least about 100,000 features/cm², there are interfeature areas between the majority of features, substantially free of oligonucleotides.

Additionally, the sequence of nucleotides in a given feature may vary based on a particular synthesis reaction. For example, while the majority of oligonucleotides in a feature may be 60 mer, some may be less than 60 mer but otherwise comprise subsequences of the 60 mer sequence. However, in one aspect, at least about 75%, at least about 80%, at least about 90%, at least about 95% of the oligonucleotides of a feature comprise identical sequences (e.g., sequences of identical base composition and length).

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces.

In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

In one embodiment, an array is synthesized using a method as described in U.S. Ser. No. 10/813,467, the entirety of which is incorporated by reference herein.

In some embodiments, previously identified regions from a particular chromosomal region of interest are used as array elements. Such regions are becoming available as a result of rapid progress of the worldwide initiative in genomics. In certain embodiments, the array can include features made up of surface immobilized oligonucleotides which “tile” a particular region (which have been identified in a previous assay), by which is meant that the features correspond to region of interest as well as genomic sequences found at defined intervals on either side of the particular region of interest, i.e., 5′ and 3′ of, the region of interest, where the intervals may or may not be uniform, and may be tailored with respect to the particular region of interest and the assay objective. In other words, the tiling density may be tailored based on the particular region of interest and the assay objective. Such “tiled” arrays and assays employing the same are useful in a number of applications, including applications where one identifies a region of interest at a first resolution, and then uses tiled arrays tailored to the initially identified region to further assay the region at a higher resolution, e.g., in an iterative protocol. Accordingly, the subject methods include at least two iterations, where the first iteration of the subject methods identifies a region of interest, and the one or more subsequent iterations assay the region with sets of tiled surface immobilized features, e.g., of increasing or alternate resolution.

Of interest are both coding and non-coding genomic regions, (as well as regions that are transcribed but not translated), where by coding region is meant a region of one or more exons that is transcribed into an mRNA product and from there translated into a protein product, while by non-coding region is meant any sequences outside of the exon regions, where such regions may include regulatory sequences, e.g., promoters, enhancers, introns, inter-genic regions, etc. In certain embodiments, one can have at least some of the features directed to non-coding regions and others directed to coding regions. In certain embodiments, one can have all of the features directed to non-coding sequences. In certain embodiments, one can have all of the features directed to, i.e., corresponding to, coding sequences.

In some embodiments, tiled oligonucleotide features, which are adjacent to each other in the genome, may be spaced at about at least 10bp, 25 bp, 50 bp, 100 bp, 150 bp, 200 bp, 300 bp, 500 bp, 750 bp, 1 kb, 2 kb, 3 kb, 4 kb, 5 kb, 6 kb, 7 kb, 8 kb, 9 kb, 10 kb or 20 kb relative to their positions in the genome. In other embodiments, adjacent tiled oligonucleotide features may be spaced at about at most 10bp, 25 bp, 50 bp, 100bp, 150 bp, 200 bp, 300 bp, 500 bp, 750 bp, 1 kb, 2 kb, 3 kb, 4 kb, 5 kb, 6 kb, 7 kb, 8 kb, 9 kb, 10 kb or 20 kb relative to their positions in the genome.

In one embodiment, at least 20%, 30%, 40%, 50%, 60%, 70%, 80%, 90% or more preferably at least 95% of the oligonucleotide features are tiled oligonucleotide features. In one embodiment, the oligonucleotide features are tiled in overlapping 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 52, 53, 54, 55, 56, 57, 58, 59, 60 or more base pair steps across the genome or portion of the genome. In a specific embodiment, the sequences of oligonucleotide features tiled at sequential sites contain overlaps, preferably at regular intervals. For example, if overlapping oligonucleotide features A, B, and C are sequentially tiled on the genome so that A is 5′ to B and C, and C is 3′ to A and B, then a portion of the 5′ end of oligonucleotide feature B will be identical to the 3′ end of oligonucleotide features A, and a portion of the 3′ end of oligonucleotide feature B will be identical to the 5′ end of oligonucleotide feature B.

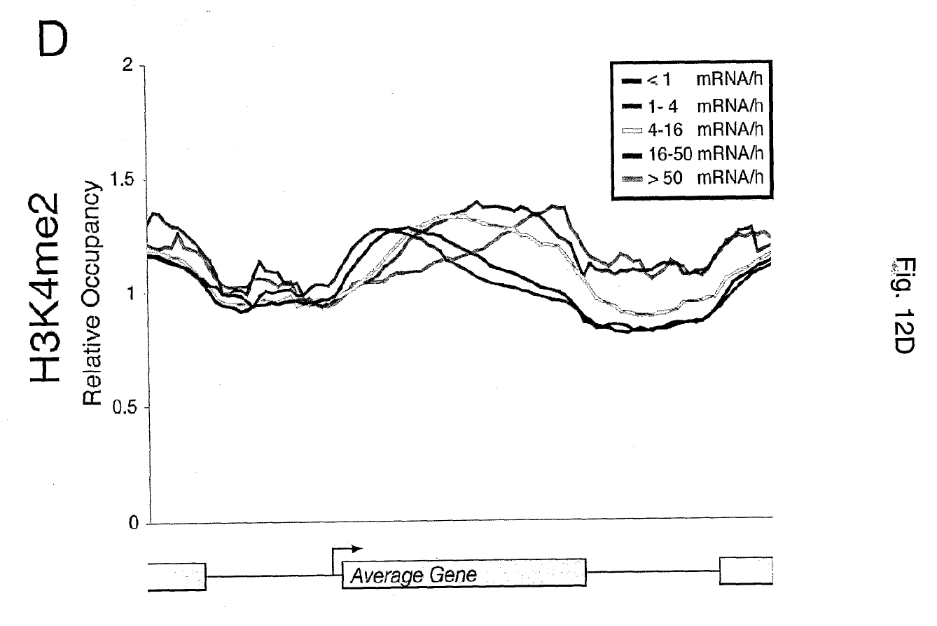

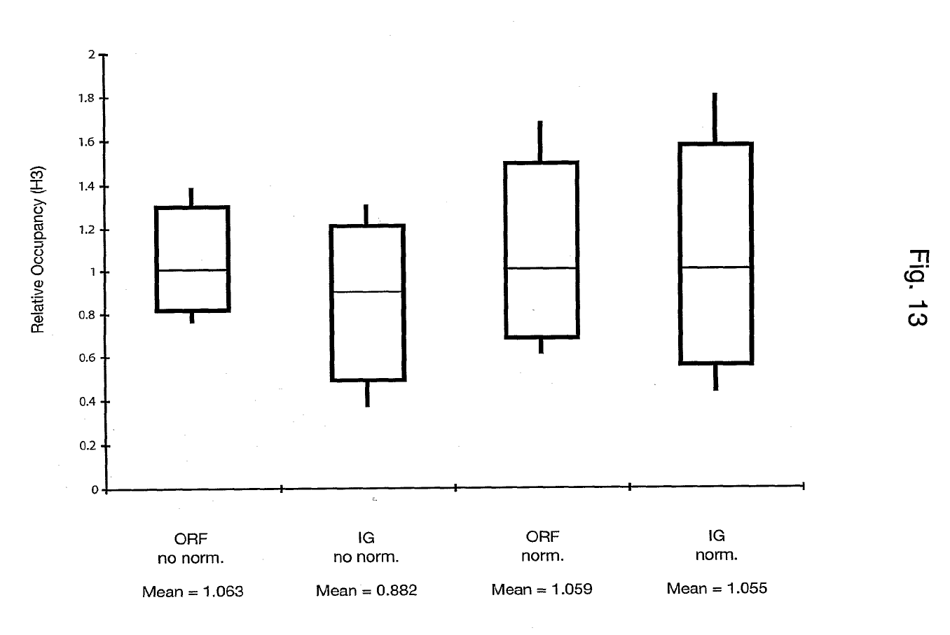

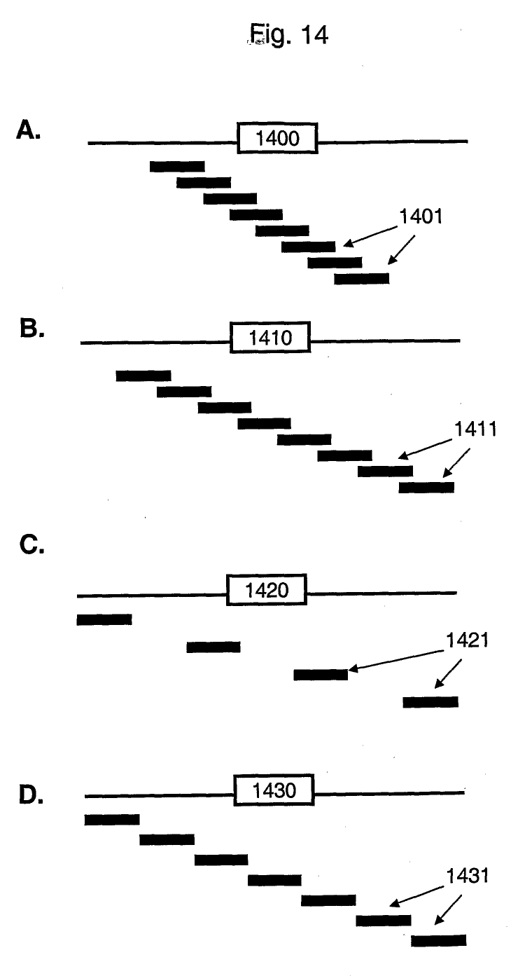

FIG. 14A-14D provides an illustration of tiled oligonucleotide features. FIG. 14A shows exon 1400 along a genome with tiled oligonucleotide features 1401. The oligonucleotide features overlap such that a tiled position on the genome in found in two adjacent probes, since each oligonucleotide feature shares half of its sequence with each adjacent probe. FIG. 14B shows another embodiment where adjacent tiled oligonucleotide features overlap by 25% so that a position in the genome is found in 1.5 of the oligonucleotide features.

In other embodiments, each oligonucleotide features shares at least 90%, 80%, 70%, 60%, 50%, 40%, 30%, 20% or 10% of its sequence with adjacent probes. In yet another embodiment, the sequences of oligonucleotide features tiled at sequential sites are spaced at intervals, preferably regular intervals, across the genome or a portion of the genome, so that a portion of genomic sequence is skipped between sequential oligonucleotide features. For example, if spaced oligonucleotide features D, E, and F are sequentially tiled on the genome so that D is 5′ to E and F, and F is 3′ to D and E, then there will be a gap in the genomic sequence between oligonucleotide feature D and oligonucleotide feature E, and between oligonucleotide feature E and oligonucleotide feature F. FIG. 14C provides an illustration of spaced oligonucleotide features 1421.

In yet another embodiment, the sequences of oligonucleotide features tiled at sequential sites are adjacent to one another, so that the oligonucleotide features are neither overlapping, nor spaced. For example, if adjacent oligonucleotide features G, H, and I are sequentially tiled on the genome so that G is 5′ to H and I, and I is 3′ to G and H, then there will be no gaps in the genomic sequence between oligonucleotide feature G and oligonucleotide feature H, or between oligonucleotide feature H and oligonucleotide feature I, and no portion of the sequence of oligonucleotide feature H will be identical to either oligonucleotide feature or oligonucleotide feature I. FIG. 14D provides an illustration of adjacent, nonoverlapping oligonucleotide features 1431.

A skilled artisan will appreciate that highly-overlapping oligonucleotide features will allow for high resolution detection of a relatively smaller portion of the genome, while less overlapping, adjacent, or spaced oligonucleotide features will provide lower resolution detection of a relatively larger portion of the genome. Preferably, oligonucleotide features are tiled so that oligonucleotide features at sequential sites overlap from a range of 10-50% of the length of the oligonucleotide feature, from 50-90% of the length of the oligonucleotide feature, or from 70-80% of the length of the oligonucleotide feature. In an alternate embodiment, for highest resolution, oligonucleotide features at sequential sites overlap at all but one base pair. The oligonucleotide features may be of different lengths to normalize binding energies of different oligonucleotides. Shorter oligonucleotide features (15-20 bp) may also provide higher resolution mapping of intron-exon boundaries.

As noted above, the “oligonucleotide feature” to which a particular probe specifically hybridizes according to the invention contains a complementary genomic polynucleotide sequence. In one embodiment, the oligonucleotide features of an array preferably consist of nucleotide sequences of no more than 1,000 nucleotides. In some embodiments, the oligonucleotide features of the array consist of nucleotide sequences of 10 to 1,000 nucleotides. In a preferred embodiment, the nucleotide sequences of the oligonucleotide features are in the range of 10-200 nucleotides in length and are genomic sequences of a species of organism, such that a plurality of different oligonucleotide features is present, with sequences complementary and thus capable of hybridizing to the genome of such a species of organism, sequentially tiled across all or a portion of such genome. In other specific embodiments, the oligonucleotide features are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, and most preferably are about 60 nucleotides in length.

In a typical example of a genome scanning array of the invention, the oligonucleotide features (e.g., 60-mers) are overlapping by X bp, where X is a selected number, preferably less than 100, 50, or 25 bp, and is for example 5, 8, 10, or 15 bp, or in the range of 5-20 bp. In an another embodiment, an array contains adjacent oligonucleotide features, or spaced oligonucleotide features with genomic sequence gaps of, for example, 10, 50, 100, 500, or 1,000 bp between the sequences complementary to sequential oligonucleotide features. A skilled artisan will appreciate that if a genomic sequence for any given oligonucleotide feature is an identical distance both 5′ and 3′ from genomic sequences for two other oligonucleotide features, either of the other oligonucleotide features has a genomic sequence “closest in the genome.” In an alternate embodiment, the screening array includes a single oligonucleotide feature for each predicted exon in the genome of the organism. In another alternate embodiment, a scanning or screening array is tested under many conditions, and clustering analysis is performed to determine which exons belong to which genes, and to identify regions for further analysis with high-resolution scanning arrays.

Depending on the data desired to be obtained, an array can be designed to have sequences tiled at larger intervals to conduct an initial survey of a large genomic region, or smaller intervals to more completely analyze a part of the genome. The highest resolution can be obtained if the oligonucleotides are tiled at single base intervals across the genome. The shortest possible oligonucleotides as oligonucleotide features consistent with obtaining reliable and specific hybridization given the complexity of the genome are desired. In a specific embodiment, the distance between 5′ ends of oligonucleotide features at sequential sites is always less than 500 bp, and more preferably always less than 250 bp, 100 bp, 50 bp, 10 bp, 5 bp, or 2 bp. In another specific embodiment, the genomic sequences for a set of oligonucleotide features on an array span a genomic region of at least 25,000 bp, 50,000 bp, and more preferably at least 75,000 bp, 200,000 bp, 500,000 bp, or 1,000,000 bp.

In one embodiment, the oligonucleotide features comprise a nucleic acid having a length ranging from about 10 to about 200 nt including from about 10 or about 20 nt to about 100 nt, where in many embodiments the immobilized nucleic acids range in length from about 50 to about 90 nt or about 50 to about 80 nt, such as from about 50 to about 70 nt. In a preferred embodiment, the nucleic acid has a length of about 60 nucleotides.

In one embodiment, the oligonucleotide features bound to a surface of a solid support includes sequences representative of locations distributed across at least a portion of a genome. In one embodiment, the oligonucleotide features have target complements spaced (uniformly or non-uniformly) throughout the genome. In one aspect, a probe set comprises probe sequences representing 47 different loci, one on each p and q arm of the 23 human chromosomes plus one locus on the Y-chromosome. In another aspect, the probe set comprises probe sequences which include repetitive sequences (e.g., such as Alu sequences, centromeric sequences, telomere sequences, LINE sequences, SINE sequences and the like). In one embodiment, the oligonucleotide features bound to a surface of a solid support samples the portion of the genome at least about every 20, 10, 5, 4, 3, 2, 1, or 0.5 kb. In one embodiment, the portion of the genome comprises at least 20%, 30%, 40%, 50%, 60%, 70%, 80%, 90%, 95% of (i) total genomic DNA; (ii) open-reading frames; (iii) promoter regions; (iv) genic regions; or (v) chromosomes. In one embodiment, the portion of the genome comprises at least 1 Mb, 2 Mb, 3 Mb, 4 Mb, 5 Mb, 10 Mb, 15 Mb, 25 Mb, 50 Mb, 100 Mb, 200 Mb, 500 Mb, 1000 Mb, 2000 Mb or 3000 Mb of genomic sequence. For example, 5,000 oligonucleotide features of about 60 nucleotides each may be used to tile a 5 Mb portion of a chromosome at every about 1 kb.

In one embodiment, at least 20%, 30%, 40%, 50%, 60%, 70%, 80%, 90% or 95% of the oligonucleotide features correspond to non-coding genomic regions. In one embodiment, at least 20%, 30%, 40%, 50%, 60%, 70%, 80%, 90% or 95% of the oligonucleotide features correspond to non-promoter regions. In one embodiment, at least 20%, 30%, 40%, 50%, 60%, 70%, 80%, 90% or 95% of the oligonucleotide features do not comprise entire reading frames or entire exons or both.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. Nos. 6,242,266, 6,232,072, 6,180,351, 6,171,797, 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

In certain embodiments of particular interest, in situ prepared arrays are employed. In situ prepared oligonucleotide arrays, e.g., nucleic acid arrays, may be characterized by having surface properties of the substrate that differ significantly between the feature and inter-feature areas. Specifically, such arrays may have high surface energy, hydrophilic features and hydrophobic, low surface energy hydrophobic interfeature regions. Whether a given region, e.g., feature or interfeature region, of a substrate has a high or low surface energy can be readily determined by determining the regions “contact angle” with water, as known in the art and further described in copending application having publication no. 2004-0241663, the disclosure of which is herein incorporated by reference. Other features of in situ prepared arrays that make such array formats of particular interest in certain embodiments of the present invention include, but are not limited to: feature density, oligonucleotide density within each feature, feature uniformity, low intra-feature background, low inter-feature background, e.g., due to hydrophobic interfeature regions, fidelity of oligonucleotide features making up the individual features, array/feature reproducibility, and the like. The above benefits of in situ produced arrays assist in maintaining adequate sensitivity while operating under stringency conditions required to accommodate highly complex samples.

Generally, nucleic acid hybridizations between the probes and the arrays comprise thefollowing major steps: (1) provision of array of surface immobilized nucleic acids or features; (2) optionally pre-hybridization treatment to increase accessibility of features, and to reduce nonspecific binding; (3) hybridization of the nucleic acid probes to the features on the solid surface, typically under high-stringency conditions; (4) post-hybridization washes to remove probes not bound in the hybridization; and (5) detection of the hybridized probes. The reagents used in each of these steps and their conditions for use vary depending on the particular application.

As indicated previously, hybridization is carried out under suitable hybridization conditions, which may vary in stringency as desired. In certain embodiments, highly-stringent hybridization conditions may be employed. The term “highly-stringent hybridization conditions” as used herein refers to conditions that are compatible to produce nucleic acid binding complexes on an array surface between complementary binding members, i.e., between immobilized features and complementary solution phase nucleic acids in a sample. Representative high-stringency assay conditions that may be employed in these embodiments are provided above.

The hybridization step may include agitation of the immobilized features and the sample of solution phase nucleic acids, where the agitation may be accomplished using any convenient protocol, e.g., shaking, rotating, spinning, and the like. Following hybridization, the surface of immobilized nucleic acids is typically washed to remove unbound nucleic acids. Washing may be performed using any convenient washing protocol, where the washing conditions are typically stringent, as described above.

Following hybridization and washing, as described above, the hybridization of the probes to the array is then detected using standard techniques so that the surface of immobilized features, e.g., array, is read. Reading of the resultant hybridized array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable devices and methods are described in U.S. Pat. No. 6,756,202 by Dorsel et al.; and U.S. Pat. No. 6,406,849, which references are incorporated herein by reference.

Arrays, however, may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere).

In the case of indirect labeling, subsequent treatment of the array with the appropriate reagents may be employed to enable reading of the array. Some methods of detection, such as surface plasmon resonance, do not require any labeling of the nucleic acids, and are suitable for some embodiments. In some embodiments, detecting the hybridization between the labeled/unlabeled probes and the nucleic acids complimentary to the genome is facilitated by contacting the complexes between the labeled or unlabeled probe and the nucleic acid on the array with a detection agent, wherein the amount of detection agent that binds to the complex is indicative of the level of hybridization. In one embodiment, the detection agent comprises an antibody or fragment thereof. In another embodiment, the detection agent comprises a dendrimer. The use of dendrimers for the detection microarray hybridization has been described in U.S. Pat. Pub. Nos. 2002/0051981 and 2002/0072060, hereby incorporated by reference in their entirety. In another embodiment, the detection agent binds to a double stranded nucleic acid selected from the group consisting of a DNA-DNA, DNA-RNA or RNA-RNA double stranded-nucleic acids.

Results from the reading or evaluating may be raw results (such as fluorescence intensity readings for each feature in one or more color channels) or may be processed results, such as obtained by subtracting a background measurement, or by rejecting a reading for a feature which is below a predetermined threshold and/or forming conclusions based on the pattern read from the array (such as whether or not a particular feature sequence may have been present in the sample, or whether or not a pattern indicates a particular condition of an organism from which the sample came).

DNA microarray and methods of analyzing data from microarrays are well-described in the art, including in DNA Microarrays: A Molecular Cloning Manual, Ed by Bowtel and Sambrook (Cold Spring Harbor Laboratory Press, 2002); Microarrays for an Integrative Genomics by Kohana (MIT Press, 2002); A Biologist's Guide to Analysis of DNA Microarray Data, by Knudsen (Wiley, John & Sons, Incorporated, 2002); and DNA Microarrays: A Practical Approach, Vol. 205 by Schema (Oxford University Press, 1999); and Methods of Microarray Data Analysis II, ed by Lin et al. (Kluwer Academic Publishers, 2002), hereby incorporated by reference in their entirety.

In certain embodiments of the methods described herein, one or more steps are performed in different locations. In one embodiment, the fragments to which the protein of interest binds are isolated in a first location, while hybridization of the probes to an array is performed in a second location. An optional step of synthesizing probes from the fragments may be performed at either location. When two locations are used, method comprises, in some embodiments, the transport of DNA fragments or probes generated therefrom from the first location to the second location. In one embodiment, the first location is remote to the second location. A remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. In one embodiment, two locations that are remote relative to each other are at least 1, 2, 3, 4, 5, 10, 20, 50, 100, 200, 500, 1000, 2000 or 5000 km apart. In another embodiment, the two location are in different countries, where one of the two countries is the United States. In one embodiment, the two locations are in two different countries where the countries are selected from United States, Australia, Japan, South Korea, India, Israel, China, Brazil, New Zealand, South Africa, Canada, Mexico, or an European country such as the U.K, Ireland, France, Germany, Spain, Portugal, Germany, Belgium, Luxemburg, Netherlands, Switzerland, Iceland, Czech Republic, Hungary, Poland, Norway, Finland, Russia, Greece or Turkey.

Some specific embodiments of the methods described herein where steps are performed in two or more locations comprise one or more steps of communicating information between the two locations. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In one specific embodiment, the methods comprises one or more data transmission steps between the locations. In one embodiment, the data transmission step occurs via an electronic communication link, such as the internet. In one embodiment, the data transmission step from the first to the second location comprises experimental parameter data, wherein the experimental parameter data comprises data selected from: (a) the phylogenetic species of the genome; (b) clinical data from the organism from which the genome was derived; and (c) a microarray to which the labeled probes are to be hybridized.

In some embodiments, the data transmission step from the second location to the first location comprises data transmission to intermediate locations. In one specific embodiment, the method comprises one or more data transmission substeps from the second location to one or more intermediate locations and one or more data transmission substeps from one or more intermediate locations to the first location, wherein the intermediate locations are remote to both the first and second locations. In another embodiment, the method comprises a data transmission step in which a result from identifying regions of a genome is transmitted from the second location to the first location.

The protein of interest may be native to the cell, or it may be a recombinant protein. By native it is meant that the protein of interest occurs naturally in the cell. In some embodiments, the transcriptional regulator is from a species which is different from that of the genome. In some embodiments, the protein of interest is a viral protein. In such embodiments, a cell having the genome may be contacted with the virus and chromatin extracted from the infected cell after allowing sufficient time for the viral proteins to be expressed. In some embodiments, a recombinant protein of interest may have missense mutations, truncations, or inserted sequences or entire domains from other naturally-occurring proteins. A tagged protein of interest may be used in some embodiments, especially when the tag facilitates its immunoprecipitation.

In certain embodiments of the invention, the protein of interest comprises specific transcription factors, coactivators, corepressors or complexes thereof. Transcription factors bind to specific cognate DNA elements such as promoters, enhancers and silencer elements, and are responsible for regulating gene expression. Transcription factors may be activators of transcription, repressors of transcription or both, depending on the cellular context. Transcription factors may belong to any class or type of known or identified transcription factor. Examples of known families or structurally-related transcription factors include helix-loop-helix, leucine zipper, zinc finger, ring finger, and hormone receptors. Transcription factors may also be selected based upon their known association with a disease or the regulation of one or more genes.

Antibodies directed to any transcriptional coactivator or corepressor may also be used according to the invention. Examples of specific coactivators include CBP, CTIIA, and SRA, while specific examples of corepressors include the mSin3 proteins, MITR, and LEUNIG. Furthermore, the genes regulated by proteins associated with transcriptional complexes, such as the histone acetylases (HATs) and histone deacetylases (HDACs), may also de determined using the methods described herein. Histone Deacetylases are described, for example, in Johnstone, R. W., “Histone-Deacetylase Ihibitors: Novel Drugs for the Treatment of Cancer”, Nature Reviews, Volume I, pp. 287-299, (2002) and PCT Publication Nos. 00/10583, 01/18045, 01/42437 and 02/08273. U.S. Patent Publication No. 2005/0159470 describes members of the three classes of histone acetylases in Tables 1-3.

In other embodiments of the methods described herein, the protein of interest is a DNA-binding protein, such as a basal transcription factor or a component of the basal transcription machinery. Exemplary components of the basal transcription machinery include RNA polymerases, including polI, polII and polIII, TBP, NTF-1 and Sp1 and any other component of TFIID, including, for example, the TAFs (e.g. TAF250, TAF150, TAF135, TAF95, TAF80, TAF55, TAF31, TAF28, and TAF20), or any other component of a polymerase holoenzyme. In one embodiment of the methods described above, the member of the transcriptional machinery is an RNA polymerase, such as RNA polymerase II, a TATA-binding protein, or any other component of TFIID, including, for example, the TAFs (e.g. TAF250, TAF150, TAF135, TAF95, TAF80, TAF55, TAF31, TAF28, and TAF20).

In some embodiments, the protein of interest is a histone. Histones are small, positively charged proteins that are rich in basic amino acids (positively charged at physiological pH), which contact the phosphate groups (negatively charged at physiological pH) of DNA. There are five main classes of histones H1, H2A, H2B, H3, and H4. Four pairs of each of H2A, H2B, H3 and H4 together form a disk-shaped octomeric protein core, around which DNA (about 140 base pairs) is wound to form a nucleosome.

The methods described herein may be applied to protein of interest that has been causally implicated in a disease. Examples of diseases and transcriptional regulators which cause them may be found in the scientific and medical literature by one skilled in the art, including in Medical Genetics, L. V. Jorde et al., Elsevier Science 2003, and Principles of Internal Medicine, 15th edition, ed by Braunwald et al., McGraw-Hill, 2001; American Medical Association Complete Medical Encyclopedia (Random House, Incorporated, 2003); and The Mosby Medical Encyclopedia, ed by Glanze (Plume, 1991). In some embodiments, the disorder is characterized by impaired function of at least one of the following organs or tissues: brain, spinal cord, heart, arteries, esophagus, stomach, small intestine, large intestine, liver, pancreas, lungs, kidney, urinary tract, ovaries, breasts, uterus, testis, penis, colon, prostate, bone, scalp, muscle, cartilage, thyroid gland, adrenal gland, pituitary, bone marrow, blood, thymus, spleen, lymph nodes, skin, eye, ear, nose, teeth or tongue.

In some embodiments of the methods described herein, the cell has been treated with an agent, such as compound or a drug, prior to the fragmenting of genomic DNA and preferably while the cell is alive. Some preferred agents include those which bind to and/or regulate the expression of transcriptional regulators, or which are suspected of doing so. In some embodiments, the regions of the genome that are bound by a given transcriptional regulator are determined both in a cell that is contacted with an agent and in a cell that is not contacted with the agent, or that is contacted with a different amount of the agent. Such methods may be used to identify compounds that alter the types of genes and/or the extent to which a transcriptional regulators controls transcription of genes. Furthermore, such approaches may be used to screen for agents which alter the activity, DNA-binding specificity or expression of a transcriptional regulator.

In one embodiment, fragmenting the genomic DNA comprises fragmenting the genomic DNA of a population of cells. In one embodiments of the methods described herein, the population of cells comprises less than 10⁸, 10⁷, 10⁶, 10⁵, 10⁴, 10³ or 10². In some embodiments, the population of cells comprises less than 10⁸, 10⁷, 10⁶, 10⁵, 10⁴, 10³ or 10² cells which express the protein of interest, but also comprises cells which do not express the protein of interest. In one embodiment, the cell population is a population that has been isolated using fluorescent-activated cell-sorting (FACS).

In one embodiment of the methods described herein, the chromatin is from primary cells. Primary cells are isolated from an organism and have undergone minimum passaging in vitro, and thus maintain most of the phenotypic characteristics of cells in the organism. In a specific embodiment, the primary cells are primary cells that have doubled less than ten times ex vivo.

In some embodiments, the chromatin is derived from transplant-grade tissue or freshly isolated tissue. In some embodiments, the cell is derived from a tissue biopsy, such as from a subject afflicted with, or suspected of being afflicted with, a disorder.

The cell type from which the chromatin is obtained may be any cell type. The cell may be an eukaryotic cell or a prokaryotic cell. Eukaryotic cells includes those from metazoans and those from single-celled organism such as yeast. In some preferred embodiments, the cell is a mammalian cell, such as a cell from a rodent, a primate or a human. The cell may be a wild-type cell or a cell that has been genetically modified by recombinant means or by exposure to mutagens. The cell may be a transformed cell or an immortalized cell. In some embodiments, the cell is from an organism afflicted by a disease. In some embodiments, the cell comprises a genetic mutation that results in disease, such as in a hyperplastic condition.

In preferred embodiments of the methods described herein, the cell populations are contained within wells of multi-well plates to facilitate parallel handling of cells and reagents. In specific embodiments, the multi-well plate has 24, 48, 96 or 384 wells. Standard 96 well microtiter plates which are 86 mm by 129 mm, with 6 mm diameter wells on a 9 mm pitch, may be used for compatibility with current automated loading and robotic handling systems. The microplate is typically 20 mm by 30 mm, with cell locations that are 100-200 microns in dimension on a pitch of about 500 microns. Methods for making microplates are described in U.S. Pat. No. 6,103,479, incorporated by reference herein in its entirety.

Microplates may consist of coplanar layers of materials to which cells adhere, patterned with materials to which cells will not adhere, or etched 3-dimensional surfaces of similarly pattered materials. For the purpose of the following discussion, the terms “well” and “microwell” refer to a location in an array of any construction to which cells adhere and within which the cells are imaged. Microplates may also include fluid delivery channels in the spaces between the wells. The smaller format of a microplate increases the overall efficiency of the system by minimizing the quantities of the reagents, storage and handling during preparation and the overall movement required for the scanning operation. In addition, the whole area of the microplate can be imaged more efficiently. Multi-well test plates used for isotopic and non-isotopic assays are well known in the art and are exemplified, for example, by those described in U.S. Pat. Nos. 3,111,489; 3,540,856; 3,540,857; 3,540,858; 4,304,865; 4,948,442; and 5,047,215.

Microfluidic devices may also be used at any of the steps of the methods described herein.

For example, Chung et al. (2004) Lab Chip.; 4(2):141-7 describe a high efficiency DNA extraction microchip was designed to extract DNA from lysed cells using immobilized beads and shaking solution, which allows extraction of as little as 10³ cells. Guijt et al. (2003) Lab Chip; 3(1):1-4 describes microfluidic devices with accurate temperature control, as might be used to cycle temperature during PCR amplification. Similarly, Liu et al. (2002) Electrophoresis.; 23(10):1531-6 teaches a microfluidic device for performing PCR amplification using as little as 12 mL of sample. Cady et al. (2003) Biosens Bioelectron. 30;19(1):59-66 describes a microfluidic device that may be used to purify DNA.

Another aspect of the invention provides a program product (i.e. software product) for use in a computer device that executes program instructions recorded in a computer-readable medium to analyze data from the array hybridization steps, to transmit array hybridization data from one location to another, or to evaluate genome-wide location data between two or more genomes, such as between a cell exposed to a drug and a control cell. Another related aspect of the invention provides kits comprising the program product or the computer readable medium, optionally with a computer system. In one embodiment, the program product comprises: a recordable medium; and a plurality of computer-readable instructions executable by the computer device to analyze data from the array hybridization steps, to transmit array hybridization from one location to another, or to evaluate genome-wide location data between two or more genomes. Computer readable media include, but are not limited to, CD-ROM disks (CD-R, CD-RW), DVD-RAM disks, DVD-RW disks, floppy disks and magnetic tape.

A related aspect of the invention provides kits comprising the program products described herein. The kits may also optionally contain paper and/or computer-readable format instructions and/or information, such as, but not limited to, information on DNA microarrays, on tutorials, on experimental procedures, on reagents, on related products, on available experimental data, on using kits, on literature, and on other information. The kits optionally also contain in paper and/or computer-readable format information on minimum hardware requirements and instructions for running and/or installing the software. The kits optionally also include, in a paper and/or computer readable format, information on the manufacturers, warranty information, availability of additional software, technical services information, and purchasing information. The kits optionally include a video or other viewable medium or a link to a viewable format on the internet or a network that depicts the use of the use of the software, and/or use of the kits. The kits also include packaging material such as, but not limited to, styrofoam, foam, plastic, cellophane, shrink wrap, bubble wrap, paper, cardboard, starch peanuts, twist ties, metal clips, metal cans, drierite, glass, and rubber.

The analysis of array hybridization data, as well as the transmission of data steps, can be implemented by the use of one or more computer systems. Computer systems are readily available. The processing that provides the displaying and analysis of image data for example, can be performed on multiple computers or can be performed by a single, integrated computer or any variation thereof. For example, each computer operates under control of a central processor unit (CPU), such as a “Pentium” microprocessor and associated integrated circuit chips, available from Intel Corporation of Santa Clara, Calif., USA. A computer user can input commands and data from a keyboard and display mouse and can view inputs and computer output at a display. The display is typically a video monitor or flat panel display device. The computer also includes a direct access storage device (DASD), such as a fixed hard disk drive. The memory typically includes volatile semiconductor random access memory (RAM).

Each computer typically includes a program product reader that accepts a program product storage device from which the program product reader can read data (and to which it can optionally write data). The program product reader can include, for example, a disk drive, and the program product storage device can include a removable storage medium such as, for example, a magnetic floppy disk, an optical CD-ROM disc, a CD-R disc, a CD-RW disc and a DVD data disc. If desired, computers can be connected so they can communicate with each other, and with other connected computers, over a network. Each computer can communicate with the other connected computers over the network through a network interface that permits communication over a connection between the network and the computer.

The computer operates under control of programming steps that are temporarily stored in the memory in accordance with conventional computer construction. When the programming steps are executed by the CPU, the pertinent system components perform their respective functions. Thus, the programming steps implement the functionality of the system as described above. The programming steps can be received from the DASD, through the program product reader or through the network connection. The storage drive can receive a program product, read programming steps recorded thereon, and transfer the programming steps into the memory for execution by the CPU. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation can be embodied on a program product.

Alternatively, the program steps can be received into the operating memory over the network.

In the network method, the computer receives data including program steps into the memory through the network interface after network communication has been established over the network connection by well known methods understood by those skilled in the art. The computer that implements the client side processing, and the computer that implements the server side processing or any other computer device of the system, can include any conventional computer suitable for implementing the functionality described herein.

### IV. Methods Using Genome-Wide Location Analysis

The methods described herein for identifying regions of a genome to which a protein of interest binds are useful to identify gradients of binding of the protein throughout the length of a gene. One aspect of the invention provides a method of identifying a gradient of binding of a protein of interest within a plurality of genes in the genome of a cell, the method comprising (i) identifying regions of the genome of the cell to which a protein of interest is bound according to the methods described herein; and (ii) comparing the frequency of binding of the protein of interest along the coding region of the plurality of genes, wherein the oligonucleotide features bound to a surface of a solid support include oligonucleotide features complementary to multiple regions within the coding regions of the plurality of genes. The multiple regions within the coding regions that are used may vary. In one embodiment, the average number of regions is at least 2, 3, 4, 5, 6, 7, 8, 9, or 10. Higher numbers of multiple regions allow for greater resolution in calculating the gradient.

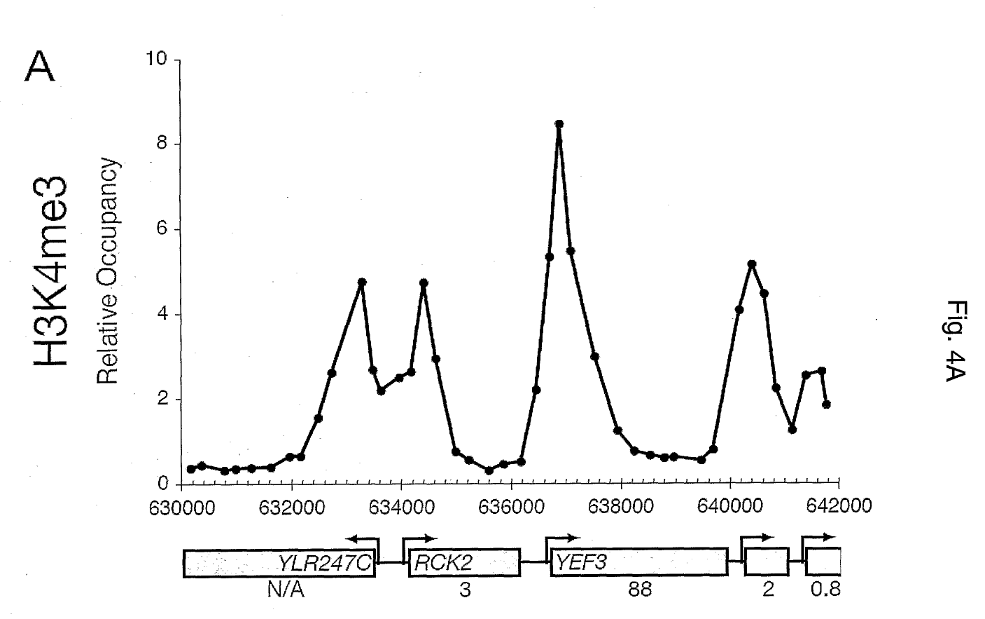

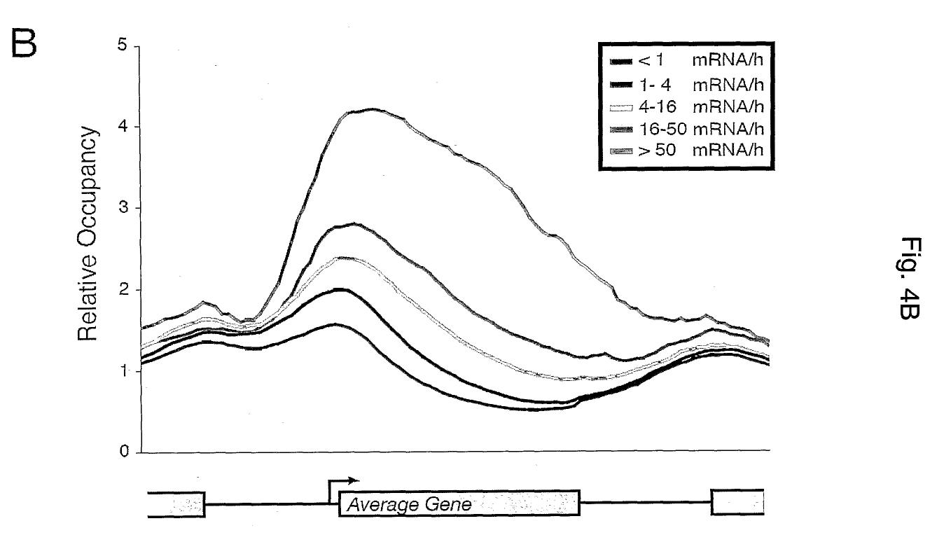

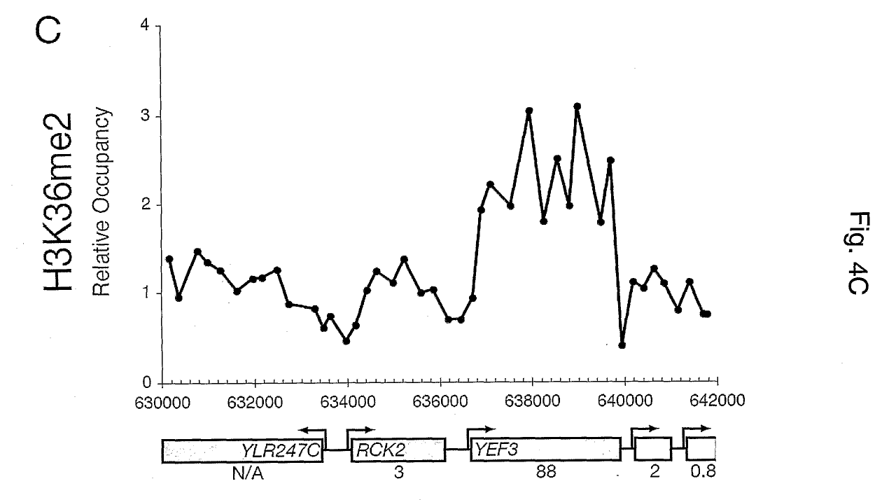

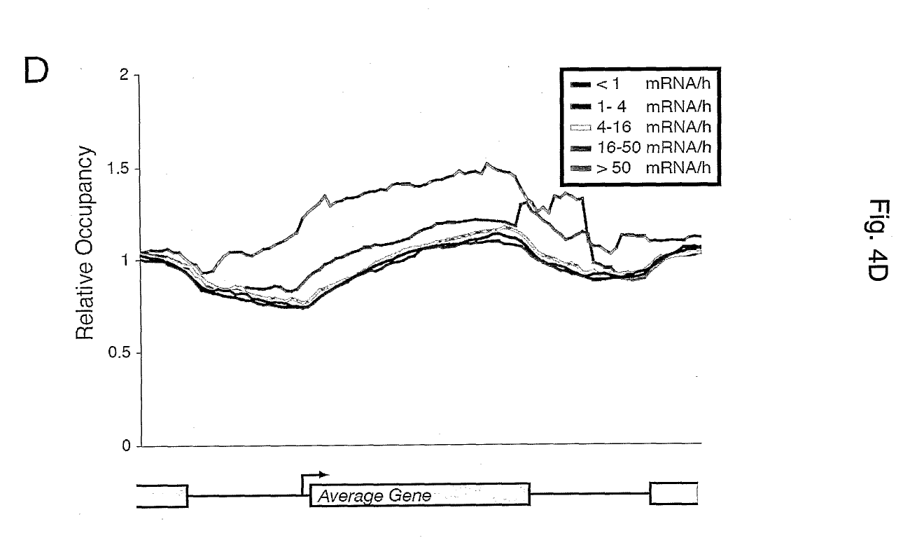

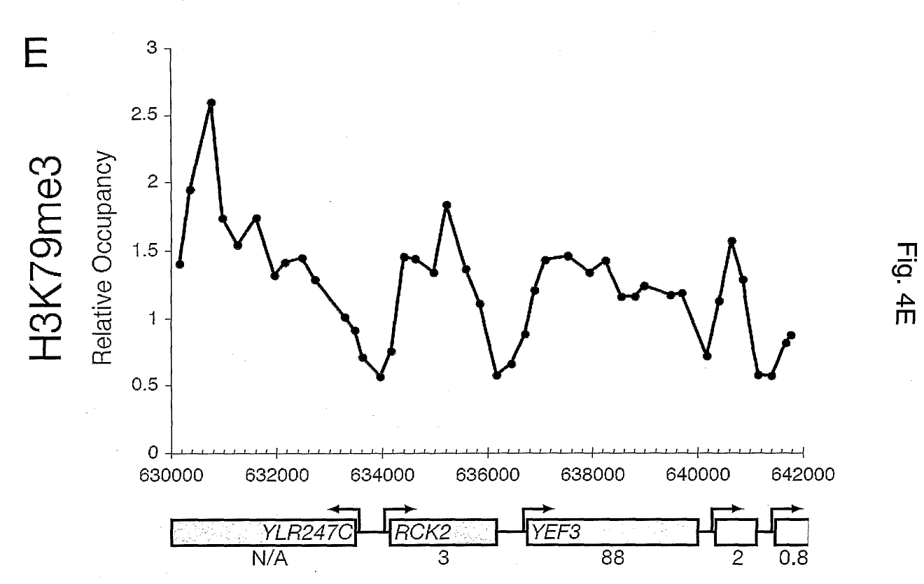

In a preferred embodiment, the array is a tiled array. In one embodiment, the plurality of genes includes at least 10, 20, 30, 40, 50, 60, 70, 80, 90, 95, 96, 97, 98, or 99% of the genes in the genome. One embodiment further comprises normalizing the length of the coding regions of the plurality of genes to identify a normalized gradient of binding along the coding regions of the genes. FIG. 4B, for example, shows a normalized gradient of H3K4 binding to an average gene.

In some embodiments of the methods described herein, the cell comprising the genome and/or the protein of interest has been treated with an agent, such as compound or a drug, prior to the fragmenting of genomic DNA, and preferably while the cell is alive.

In some embodiments of the methods described herein, the regions of the genome that are bound by a given transcriptional regulator are determined in both a cell that is contacted with an agent and in a cell that is not contacted with the agent, or that is contacted with a different amount of the agent. Such methods may be used to identify compounds that alter the types of genes and/or the extent to which a transcriptional regulators control transcription of genes. Furthermore, such approaches may be used to screen for agents which alter the activity, DNA-binding specificity or expression of a transcriptional regulator.

In some embodiments of the methods described herein, the experimental agent or drug comprises a small molecule drug, an antisense nucleic acid, an antibody, a peptide, a ligand, a fatty acid, a hormone or a metabolite. Exemplary compounds that may be used as experimental agents (e.g., a single compound, a combination of two or more compounds, a library of compounds) include nucleic acids, peptides, polypeptides, peptidomimetics, antibodies, antisense oligonucleotides, RNAi constructs (including siRNAs), ribozymes, chemical compounds, and small organic molecules. Compounds may be screened individually, in combination, or as a library of compounds. The assays described herein may also be used to screen a library of compounds to test the activity of each library member on the DNA-binding properties of protein of interest. Library members may be produced and/or otherwise generated or collected by any suitable mechanism, including chemical synthesis in vitro, enzymatic synthesis in vitro, and/or biosynthesis in a cell or organism. Chemically and/or enzymatically synthesized libraries may include libraries of compounds, such as synthetic oligonucleotides (DNA, RNA, peptide nucleic acids, and/or mixtures or modified derivatives thereof), small molecules (about 100 Da to 10 KDa), peptides, carbohydrates, lipids, and/or so on. Such chemically and/or enzymatically synthesized libraries may be formed by directed synthesis of individual library members, combinatorial synthesis of sets of library members, and/or random synthetic approaches. Library members produced by biosynthesis may include libraries of plasmids, complementary DNAs, genomic DNAs, RNAs, viruses, phages, cells, proteins, peptides, carbohydrates, lipids, extracellular matrices, cell lysates, cell mixtures, and/or materials secreted from cells, among others. Library members may be contact arrays of cell populations singly or as groups/pools of two or more members.

### EXEMPLIFICATION

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other DNA microarrays, transcriptional regulators, cell types, antibodies, ChIP conditions, or data analysis methods, all without limitation, can be employed, without departing from the scope of the invention as claimed.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999; and PCR Protocols, ed. by Bartlett et al., Humana Press, 2003.

Various publications, patents, and patent publications are cited throughout this application the contents of which are incorporated herein by reference in their entirety.

### Summary of Experimental Results

Eukaryotic genomes are packaged into nucleosomes whose position and chemical modification state can profoundly influence regulation of gene expression. Applicants profiled nucleosome modifications across the yeast genome using chromatin immunoprecipitation coupled with DNA microarrays to produce high-resolution genome-wide maps of histone acetylation and methylation. These maps take into account changes in nucleosome occupancy at actively transcribed genes, and in doing so, revise previous assessments of the modifications associated with gene expression. Both acetylation and methylation of histones are associated with transcriptional activity, but the former occurs predominantly at the beginning of genes whereas the latter can occur throughout transcribed regions. Most notably, specific methylation events are associated with the beginning, middle and end of actively transcribed genes. These maps provide the foundation for further understanding the roles of chromatin in gene expression and genome maintenance.

### Results and Discussion

### High-Resolution Genome-Wide ChIP-Chip

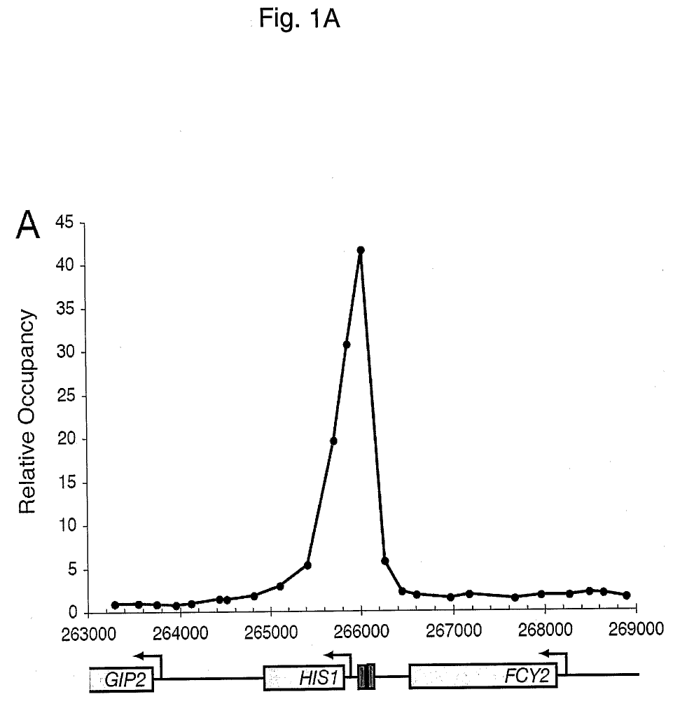

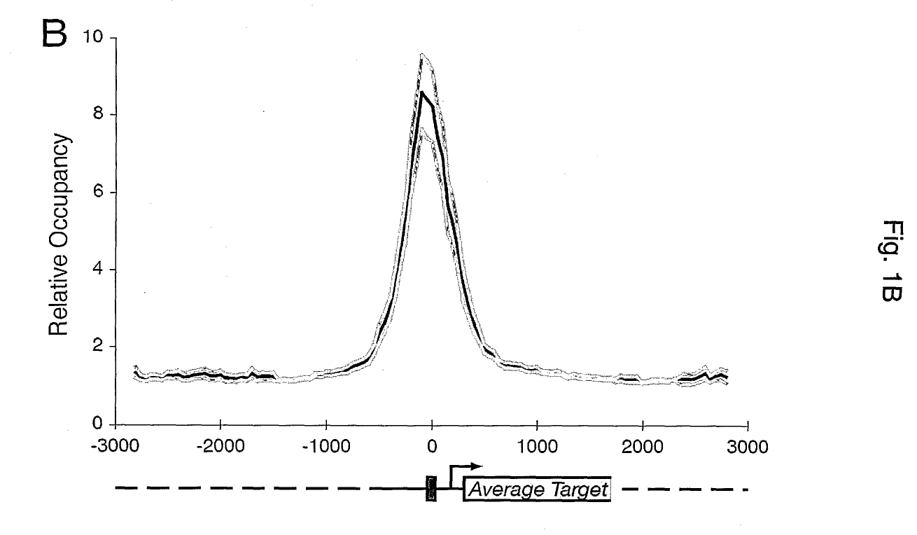

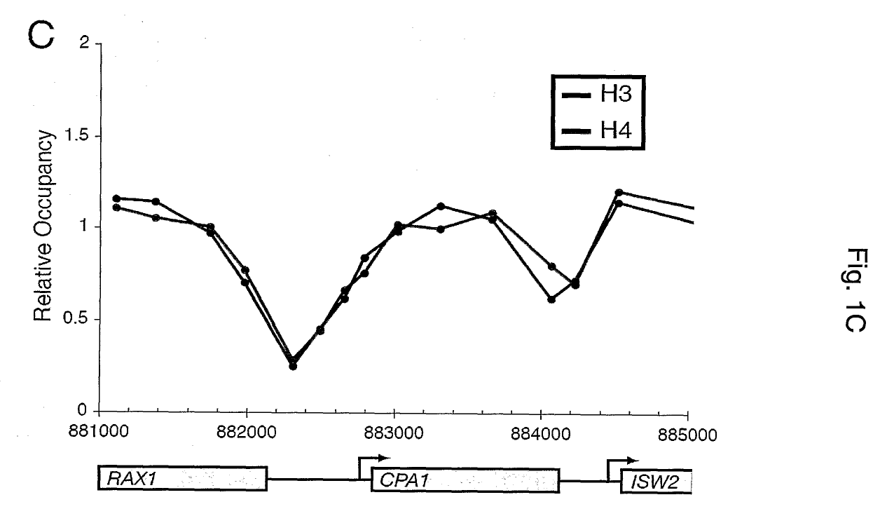

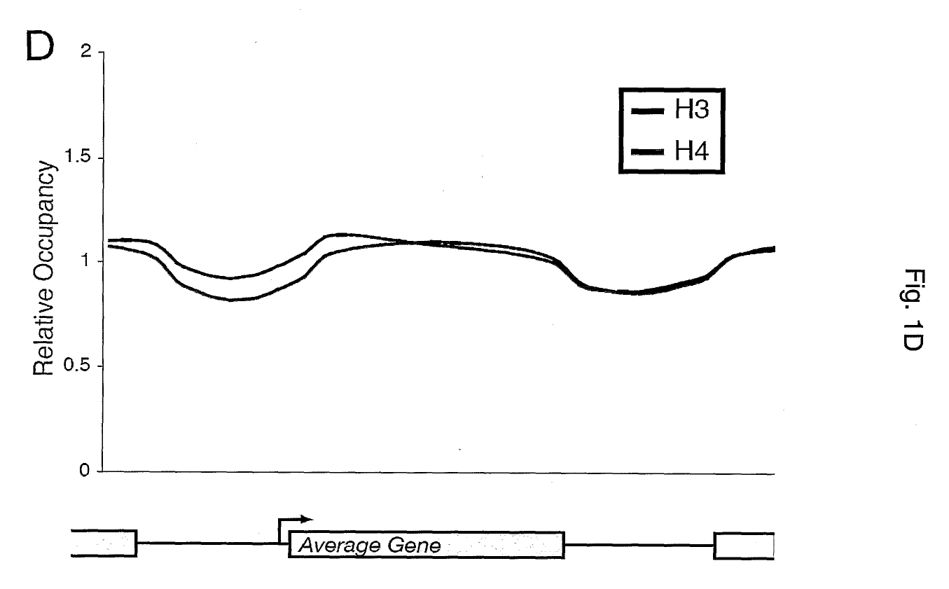

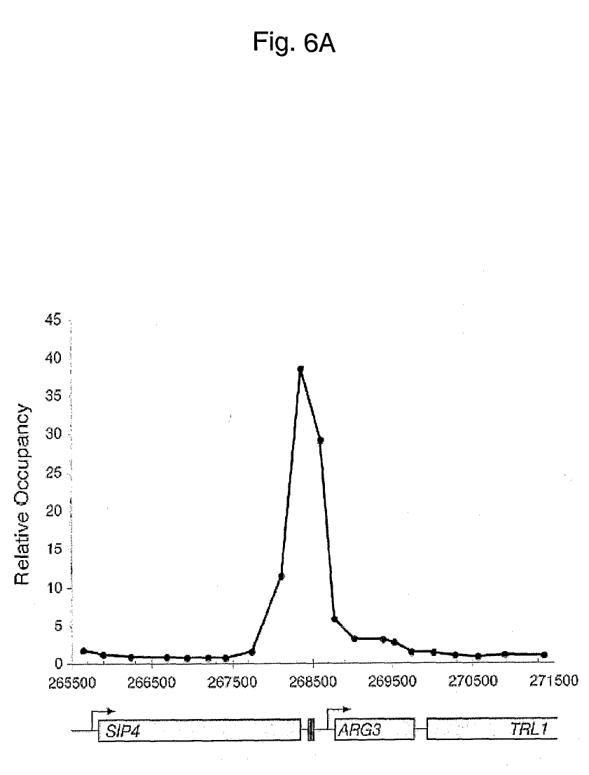

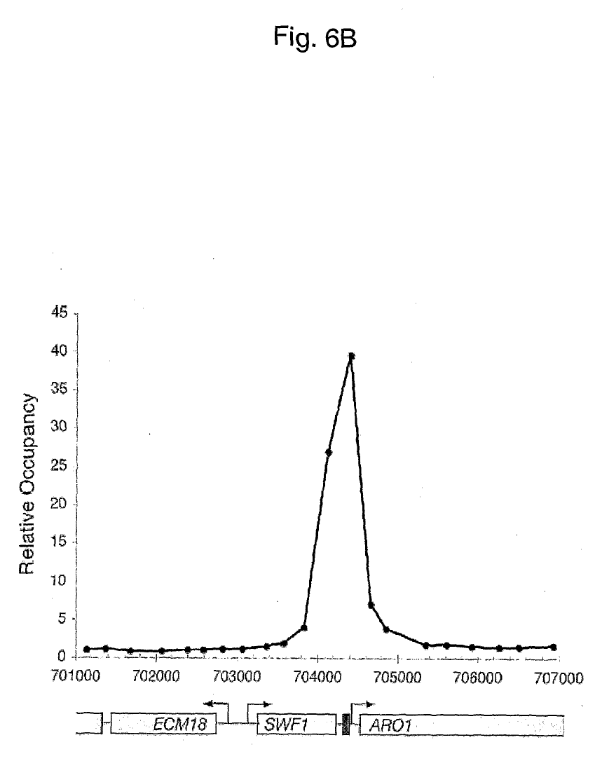

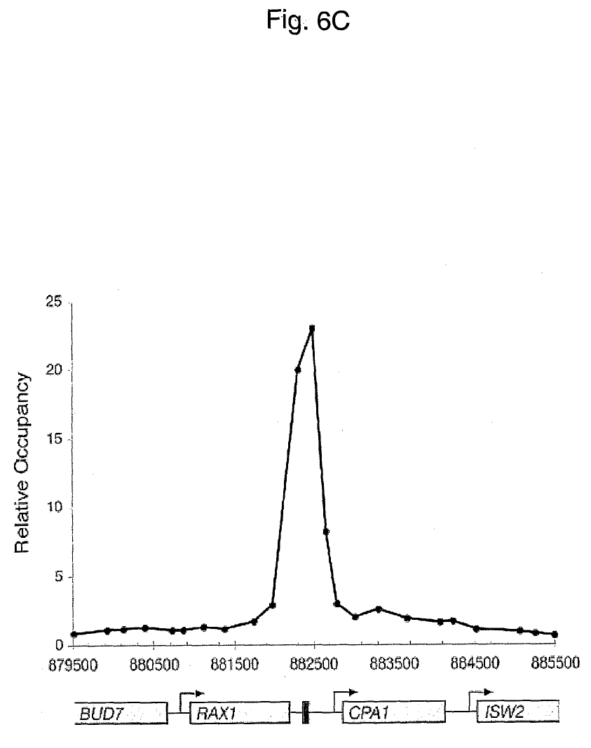

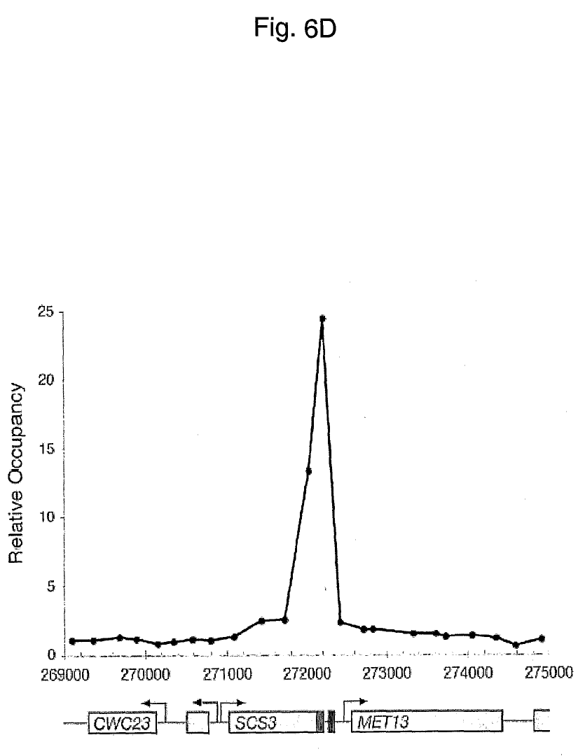

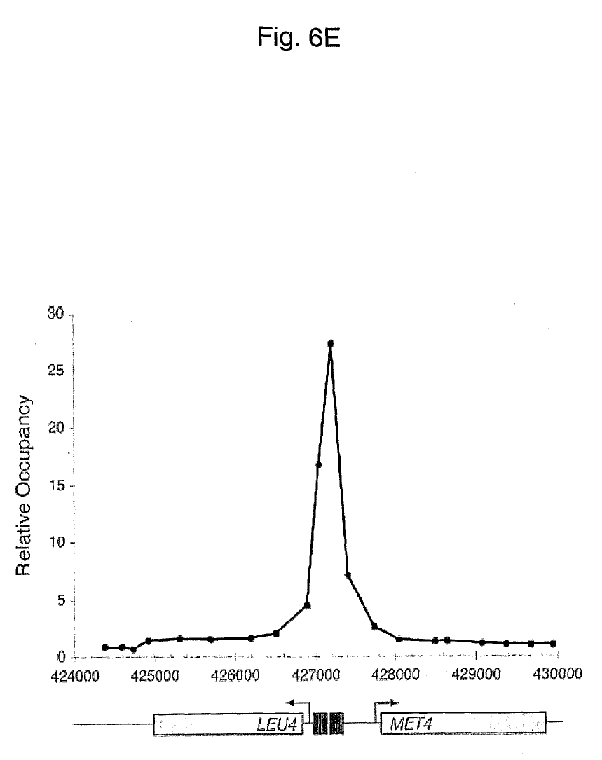

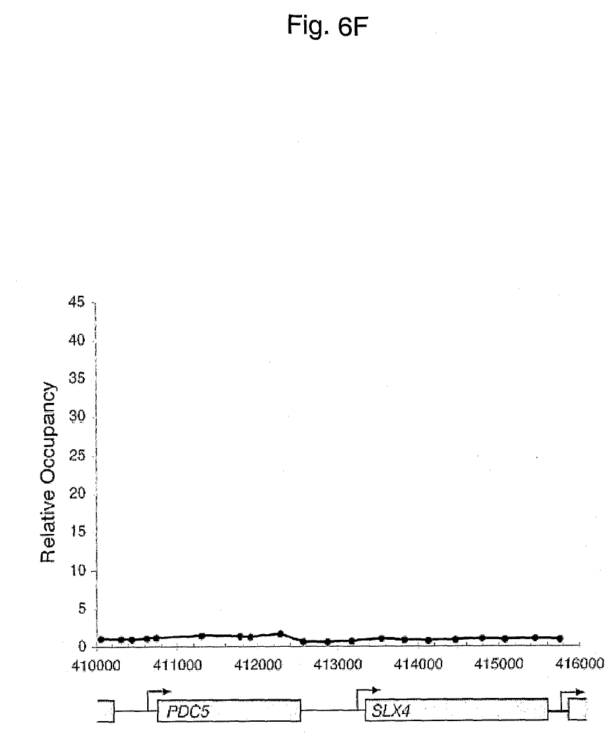

To increase the resolution and accuracy of genome-wide location analysis, Applicants designed a DNA microarray that contains over 40,000 probes for the yeast genome and developed hybridization methods that maximized signal-to-noise ratios on this array (Experimental Procedures). To test whether these modifications improved the resolution and accuracy of genome-wide binding analysis, Applicants explored the genome-wide occupancy of Gcn4, a transcriptional regulator of amino acid biosynthetic genes with a well-characterized DNA-binding specificity (Hope and Struhl, 1985; Oliphant et al., 1989), crystal structure (Ellenberger et al., 1992; O'Shea et al., 1991) and previously identified target genes (Arndt and Fink, 1986; Natarajan et al., 2001). The binding data for individual target genes are shown in FIG. 1A and in FIG. 6. For those regions for which there is strong evidence for Gcn4 binding, Applicants found that the peak of Gcn4 binding occurred directly over that binding site.

To test the accuracy of the new method, Applicants identified a test set of 84 genes most likely to be targeted by Gcn4 in vivo (FIG. 1B, Table 1, Experimental Procedures) and a set of 945 genes least likely to be targeted by Gcn4; the selection criteria for these sets of genes is described in Experimental Procedures. Based on these positive and negative Gcn4 targets, analysis of Gcn4 binding with the new method suggests a false positive rate of less than 1% and a false negative rate of ˜25%, corresponding with a total of 210 genes whose promoters are bound within the optimal P value threshold of 6×10-6 (Experimental Procedures). These results demonstrate that the new array and protocol modifications provide substantially higher resolution and accuracy than our previous method using self-printed arrays (Harbison et al., 2004; Lee et al., 2002).

TABLE 1
The following shoes the known Targets of Gcn4 Binding.

| Gene promoter | P value |
|---|---|
| ADE3 | 3.80E−07 |
| ADH5 | 4.86E−06 |
| ALD5 | 3.30E−12 |
| APG13 | 8.59E−04 |
| ARG1 | 6.44E−15 |
| ARG2/YJL072C | 4.55E−05 |
| ARG3 | 4.77E−15 |
| ARG4 | 5.22E−15 |
| ARG5 | 7.06E−14 |
| ARG8 | 1.72E−14 |
| ARO1 | 5.97E−14 |
| ARO4 | 1.78E−10 |
| ARO8 | 1.73E−05 |
| ATR1 | 5.78E−04 |
| BAP2 | 1.92E−10 |
| BNA1 | 7.04E−13 |
| CPA1 | 1.84E−14 |
| CPA2/YJR110W | 2.22E−15 |
| CPT1/YNL129W | 4.13E−04 |
| DBF20 | 1.07E−03 |
| DED81 | 1.34E−10 |
| ECM40 | 1.71E−14 |
| ESBP6/YNL124W | 8.02E−11 |
| FOL2 | 3.01E−09 |
| GAT1 | 4.65E−13 |
| HIS1 | 2.28E−14 |
| HIS3/PET56 | 5.85E−07 |
| HIS7 | 6.84E−06 |
| HOM3 | 7.88E−15 |
| HRB1 | 2.13E−11 |
| HSP78 | 3.25E−08 |
| ICY2 | 1.20E−13 |
| IDP1 | 8.75E−07 |
| ILV1 | 1.03E−02 |
| ILV3 | 4.04E−09 |
| ISU1 | 2.63E−10 |
| LEU3 | 2.26E−08 |
| LEU4 | 2.13E−14 |
| LYS1 | 2.03E−09 |
| LYS14 | 6.81E−10 |
| LYS2 | 1.06E−08 |
| MAS2/THR1 | 7.78E−04 |
| MET13 | 1.51E−11 |
| MET22 | 5.75E−06 |
| NCE103 | 3.43E−08 |
| ODC2 | 3.29E−12 |
| ORT1 | 4.28E−10 |
| PHO8 | 1.65E−12 |
| PYC2 | 9.03E−08 |
| RIM101 | 2.17E−04 |
| SFT2 | 2.16E−08 |
| SNO1/SNZ1 | 2.42E−04 |
| STB4 | 3.66E−02 |
| STR3 | 3.32E−13 |
| TEA1 | 5.51E−14 |
| THR4 | 4.32E−04 |
| TRP2 | 5.64E−07 |
| TRP4 | 1.13E−07 |
| UGA3 | 8.45E−14 |
| YBR043C | 2.70E−12 |
| YBR147W | 5.89E−06 |
| YDL054C | 9.37E−04 |
| YDR341C | 9.74E−06 |
| YGL059W | 4.66E−05 |
| YGL117W | 5.73E−09 |
| YGL186C | 5.85E−03 |
| YHR122W | 5.63E−02 |
| YHR162W | 3.38E−12 |
| YIL056W | 1.23E−12 |
| YJL200C | 4.70E−09 |
| YLR152C | 2.61E−09 |
| YMC1 | 1.30E−06 |
| YMC2 | 4.26E−07 |
| YML076C | 8.04E−04 |
| YMR135C | 2.62E−14 |
| YOL119C | 4.25E−04 |
| YPL264C | 2.63E−06 |

¹Genes were selected as targets of Gcn4 regulation because they met the following criteria: they displayed a Gcn4-dependent change in expression upon amino acid starvation (Natarajan et al., MCB, 2001); they contained within their promotersa phylogenetically conserved match to a Gcn4 binding site motif; and there was prior evidence for in vitro Gcn4 binding to their promoters. Slashes denote a shared promoter. The P value is the lowest for any probe within the corresponding promoterregion for Gcn4 binding on the current array platform.

### Global Nucleosome Occupancy

The improved accuracy and resolution of this ChIP-Chip method was used to investigate nucleosome occupancy and modification throughout the yeast genome. When Applicants examined histone occupancy with antibodies against core histone H3 or histone H4, using genomic DNA as the reference channel, Applicants found a relatively high density of nucleosomes over transcribed regions and a lower density over intergenic regions (FIG. 1C, D). FIG. 1C shows a stereotypical example of histone occupancy at a portion of chromosome XV. FIG. 1D presents composite profiles of histone H3 and H4 for 5,324 genes aligned according to the location of translation initiation and termination sites. There was a ˜20% reduction in histone occupancy in intergenic sequences relative to genic sequences for the average gene. These results are consistent with previous observations (Lee et al., 2004) and suggest that the majority of yeast genes have higher nucleosome density over transcribed regions relative to intergenic regions.

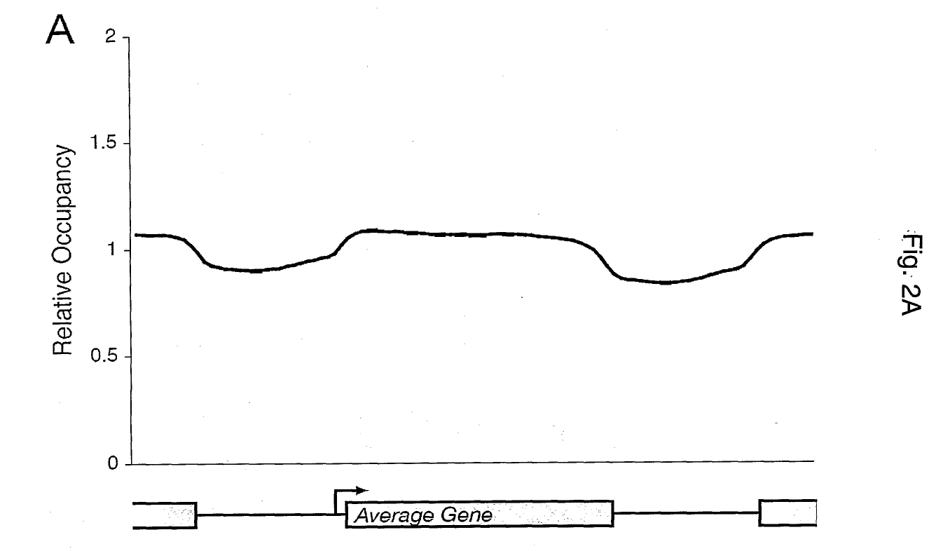

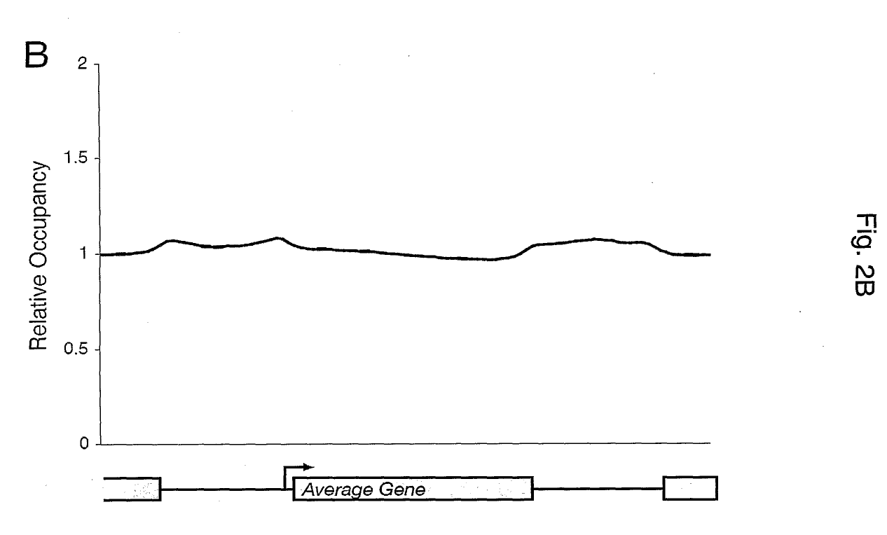

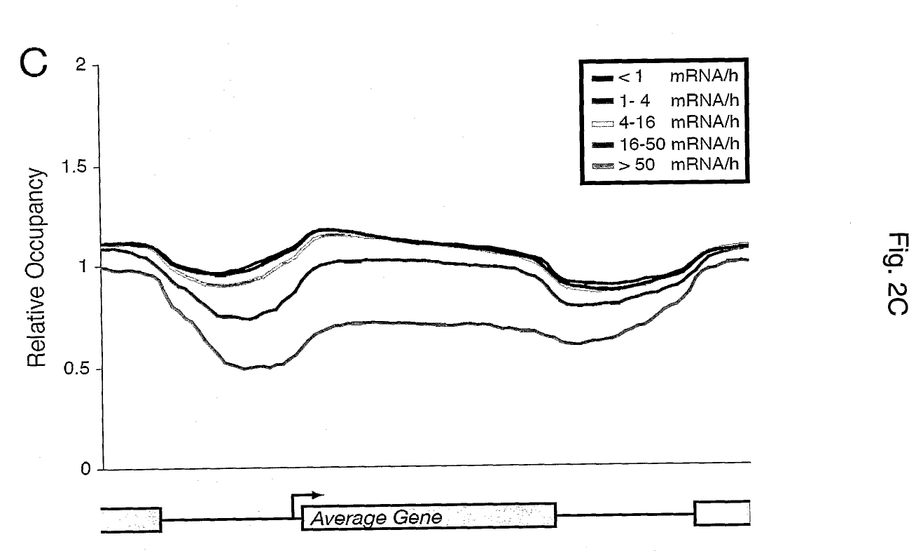

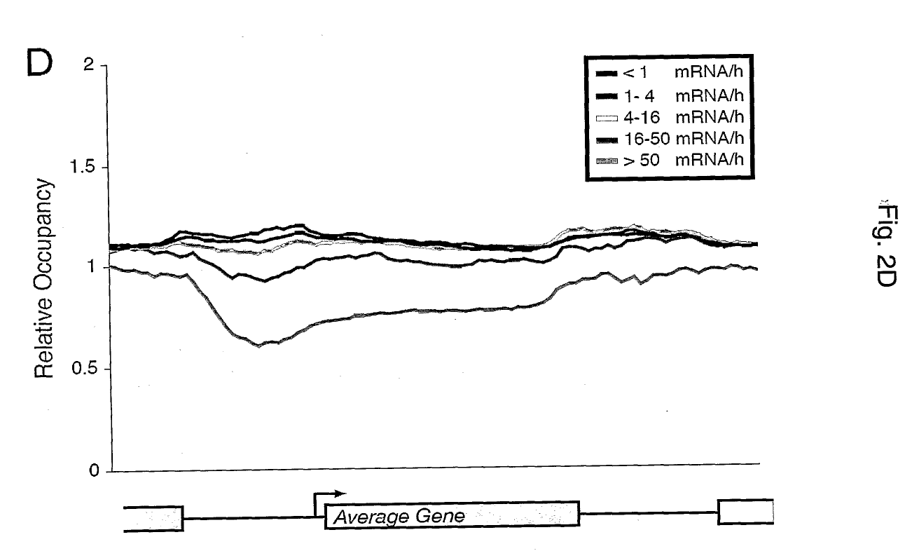

Applicants were surprised to find that differential enrichment of intergenic and genic regions also occurred in control experiments lacking antibody (compare FIG. 2A and FIG. 1D). Results similar to those in FIG. 2A were obtained in control experiments when ChIPs were performed with antibodies directed against non-histone proteins. Others have noted that different relative levels of intergenic and genic DNA are recovered using various extraction strategies (Nagy et al., 2003), but control data of this type has not yet been used to normalize the results of histone CHIP studies (Bernstein et al., 2005; Bernstein et al., 2004; Kurdistani et al., 2004; Lee et al., 2004). When these control experiments were used to normalize the histone H3 data, Applicants found that there were not substantial differences in the relative levels of intergenic versus genic DNA at the average gene (FIG. 2B). Nonetheless, approximately 40% of yeast promoters do have lower levels of histones than their downstream transcribed regions, even after the normalization by control experiments (FIG. 7), and Applicants show below that these are associated with transcribed genes.

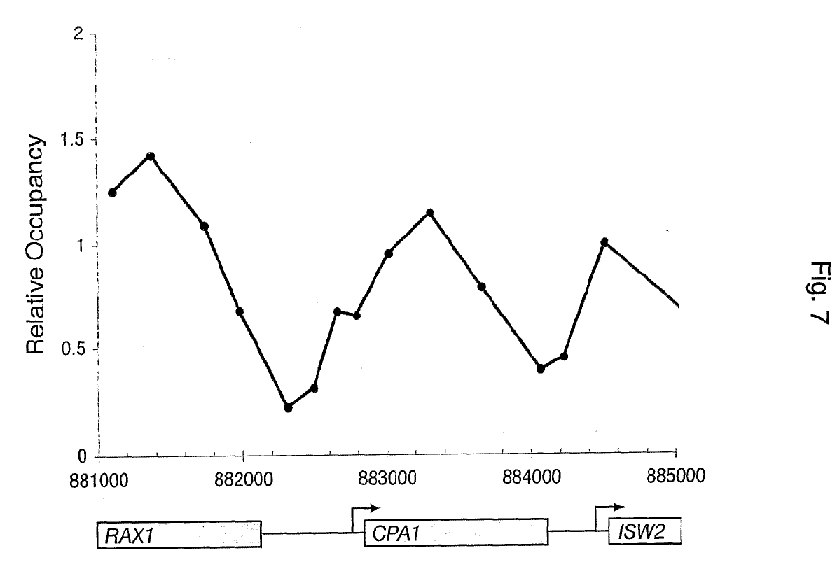

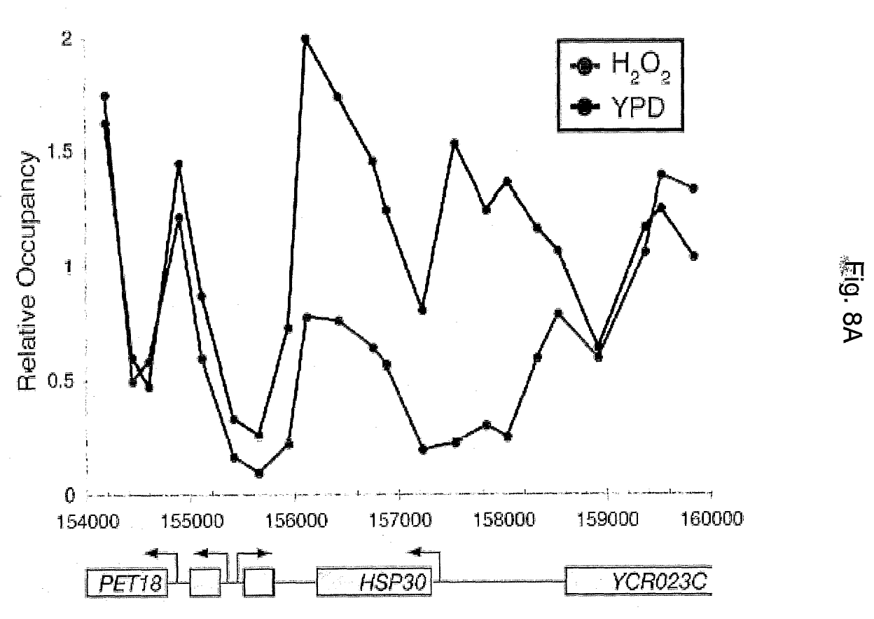

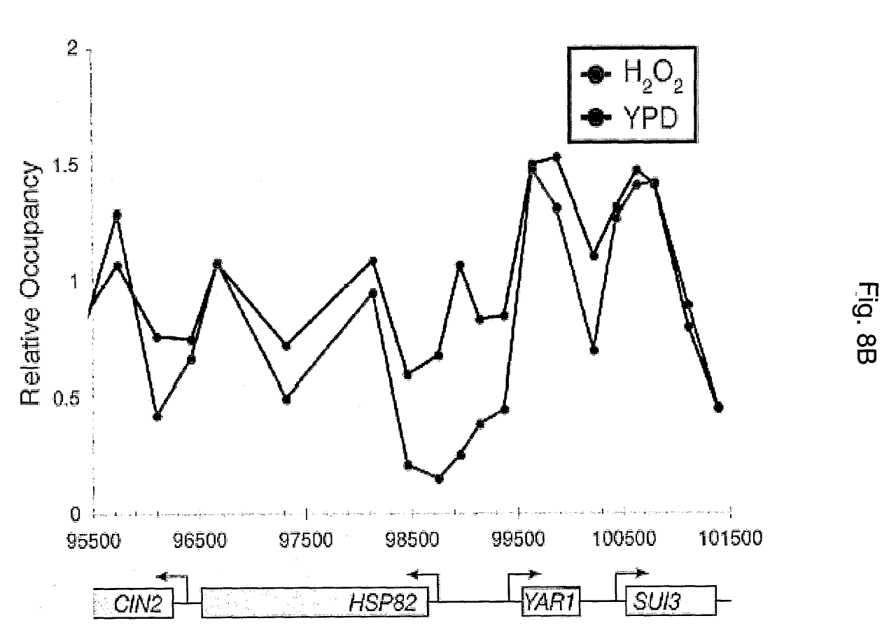

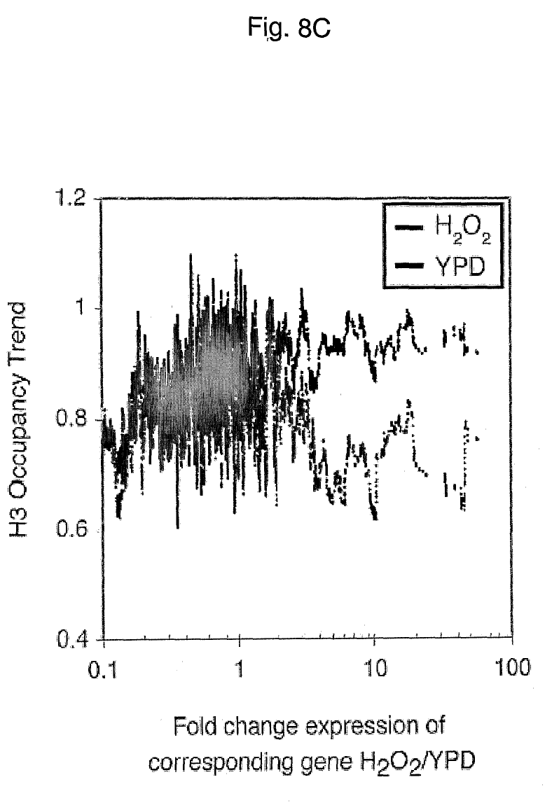

To examine the relationship between gene expression and nucleosome occupancy, Applicants assigned genes into five different classes depending on their transcriptional rate (Holstege et al., 1998) and created a composite histone H3 profile for each class (FIG. 2C, D). The composite histone profile in FIG. 2C was generated by using whole genomic DNA in the reference channel, and that in FIG. 2D was generated by normalizing to a no-antibody control CHIP. The results confirm that nucleosome occupancy at both promoter and transcribed regions inversely correlates with gene activity in either profile, in agreement with previous gene-specific and genome-wide studies (Bernstein et al., 2004; Boeger et al., 2003; Lee et al., 2004; Reinke and Horz, 2003). The results shown in FIG. 2D also suggest that nucleosome occupancy is reduced maximally at the promoters of active genes. In contrast, the promoters of transcriptionally inactive genes are as densely populated with nucleosomes as genic regions. If gene activation leads to reduced nucleosome occupancy, then dynamic activation of specific genes should cause reduced histone levels at these newly transcribed genes. To test this notion, Applicants performed ChIP-Chip with histone antibodies on cells before and after exposure to oxidative stress (Causton et al., 2001). At genes known to be activated by oxidative stress (e.g., HSP30 and HSP82), nucleosome occupancy dropped substantially (FIG. 8). These results confirm that gene activation leads to reduced nucleosome density in both promoter and transcribed regions, with the greatest effect occurring at the promoter.

### Histone Acetylation

The histone acetylases Gcn5 and Esa1 are generally recruited to the promoter regions of active genes (Robert et al., 2004) and thus Applicants would expect that the amino acid residues that are substrates of these HATs would be found preferentially acetylated at active genes. A recent genome-wide study, however, reported little correlation between transcriptional activity and acetylation of the histone H3 and H4 amino acid residues targeted by Gcn5 and Esa1 (Kurdistani et al., 2004). To understand the source of these discrepancies, Applicants used the new methods to investigate selected histone modifications genome-wide.

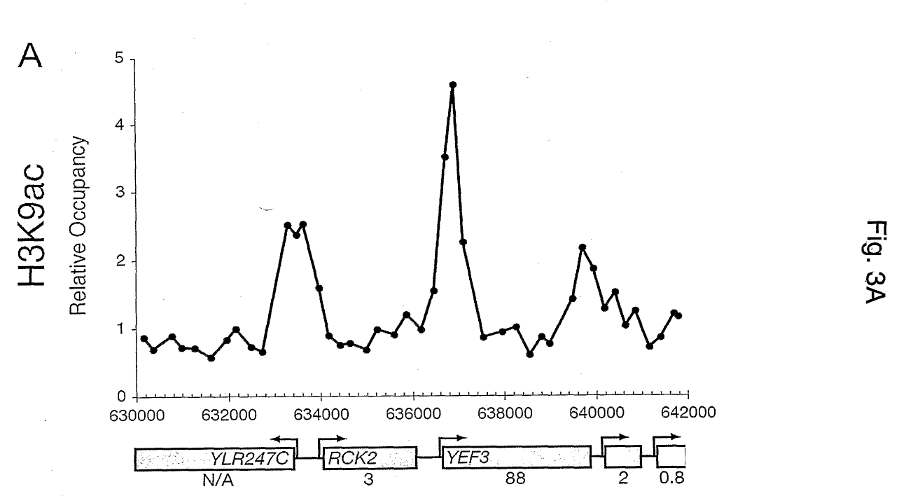

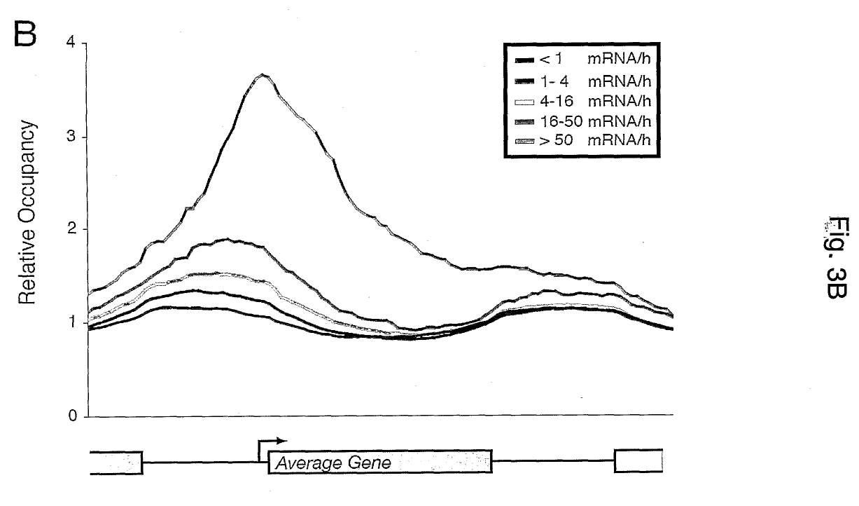

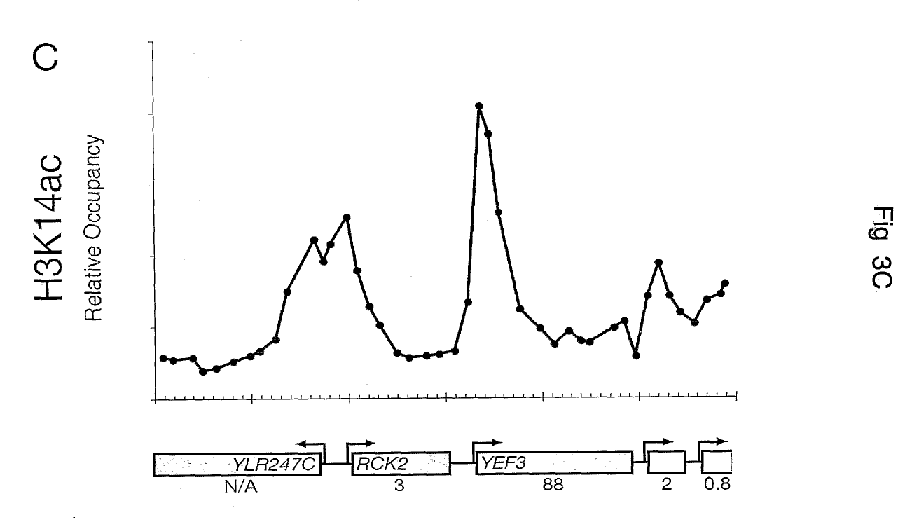

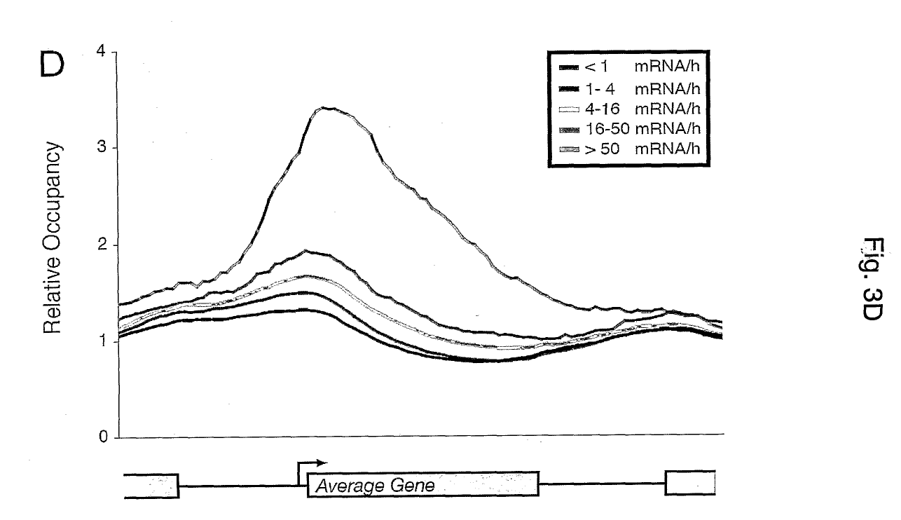

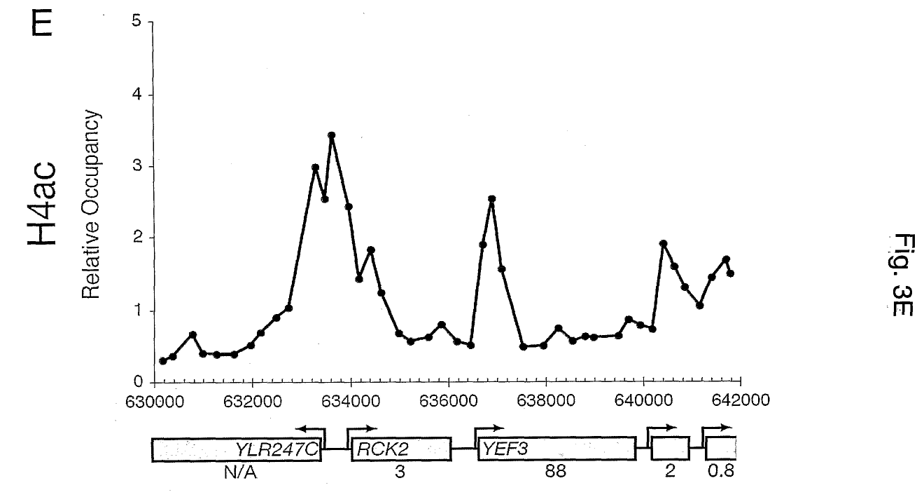

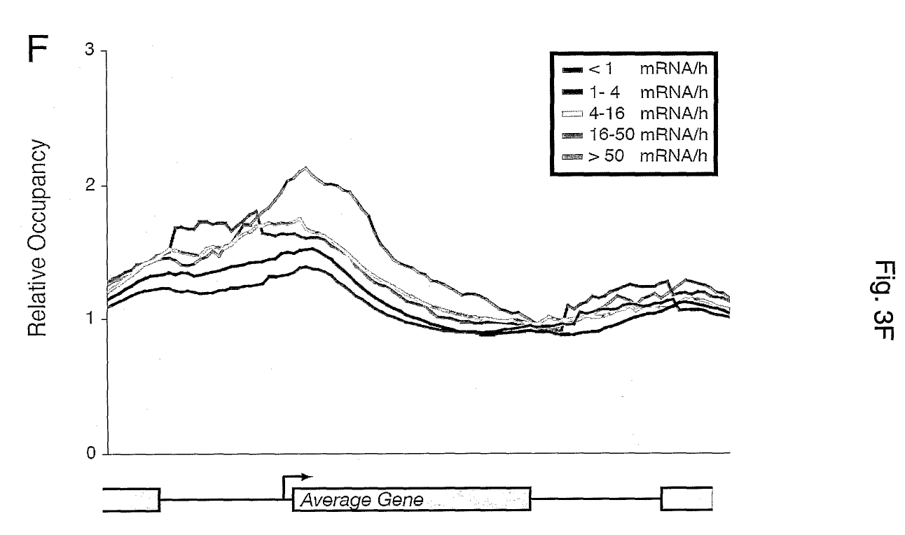

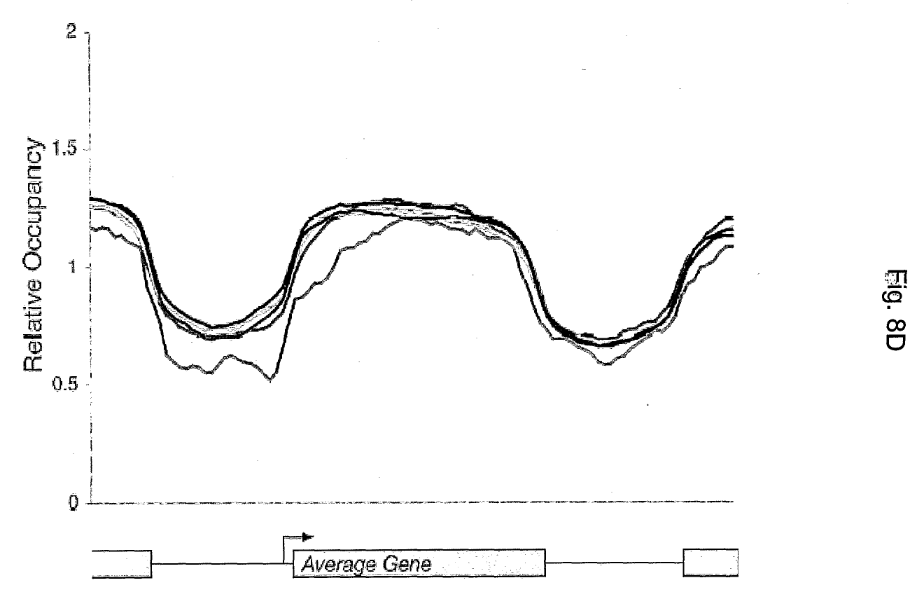

Histone H3 lysine 9 acetylation (H3K9ac) and histone H3 lysine 14 acetylation (H3K14ac) are among the modifications catalyzed by Gcn5 (Kuo et al., 1996; Utley et al., 1998; Zhang et al., 1998). Applicants used ChIP-Chip to measure the levels of histone H3 lysine 9 acetylation (H3K9ac) relative to the levels of core histone H3 genome-wide. The results show that acetylation of histone H3 at lysine 9 peaks at the predicted transcriptional start sites of active genes (FIG. 3A) and that this modification correlates with transcription rates genome-wide (FIG. 3B). Applicants also found that acetylation of histone H3 at lysine 14 peaks over the start sites of active genes (FIG. 3C) and correlates with transcription rates genome-wide (FIG. 3D). Applicants conclude that there is a positive association between Gcn5, the modifications known to be catalyzed by Gcn5, and transcriptional activity (FIG. 3 and FIG. 9A).

Four lysine residues of histone H14 are acetylated by Esal, an acetyltransferase associated with the NuA4 complex (Allard et al., 1999; Clarke et al., 1999; Vogelauer et al., 2000). Applicants measured the levels of hyperacetylated histone H4 relative to core histone genome-wide using CHIP-Chip with an antibody that recognizes histone H4 acetylated at lysines 5, 8, 12 and 16 (H4K5ac8ac12ac16ac). The results showed that H4 hyperacetylation peaks over the start sites of active genes (FIG. 3E) and correlates with transcription rates (FIG. 3F), although the association is not as strong as that observed for H3K9ac and H3K14ac. Our analysis cannot exclude the possibility that acetylation of individual lysine residues in N-terminal tail of histone H4 might correlate differently with transcriptional activity. Nonetheless, our data reveal a positive, albeit modest, correlation between Esa1 occupancy, the modifications known to be catalyzed by this enzyme, and transcriptional activity (FIG. 3 and FIG. 9B).

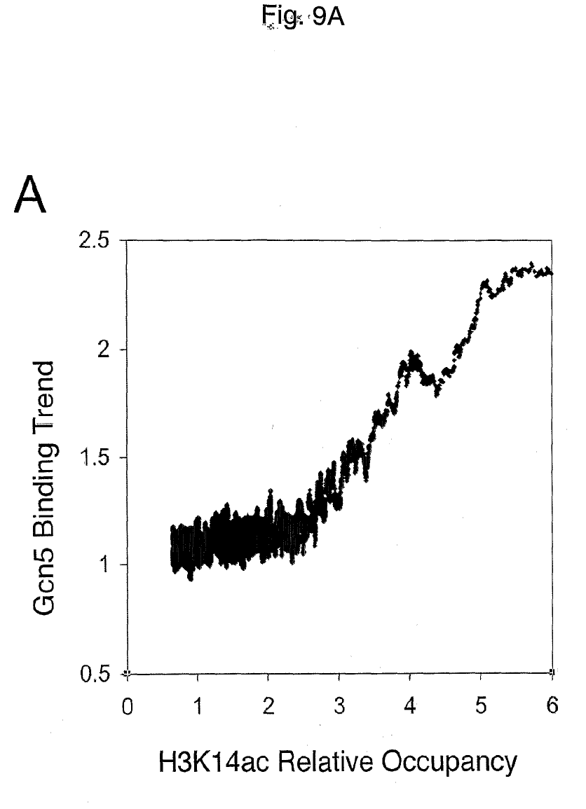

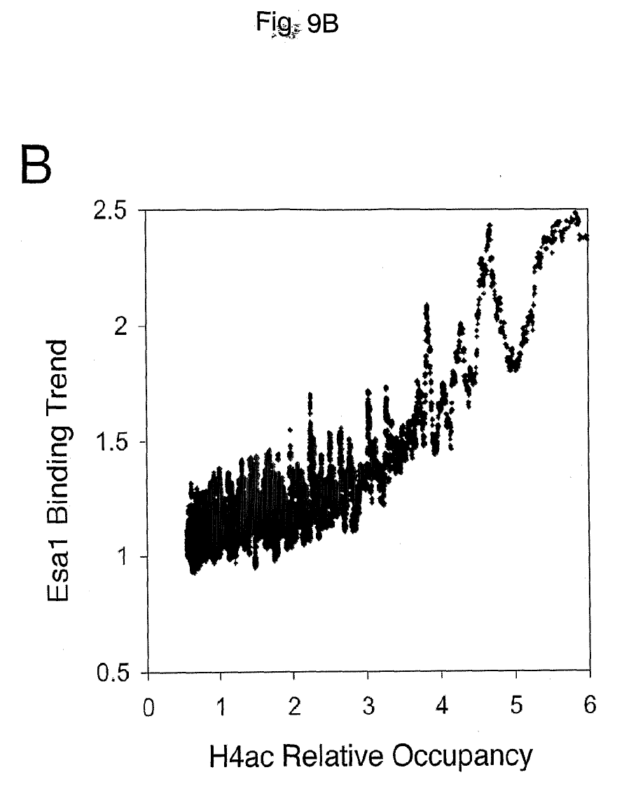

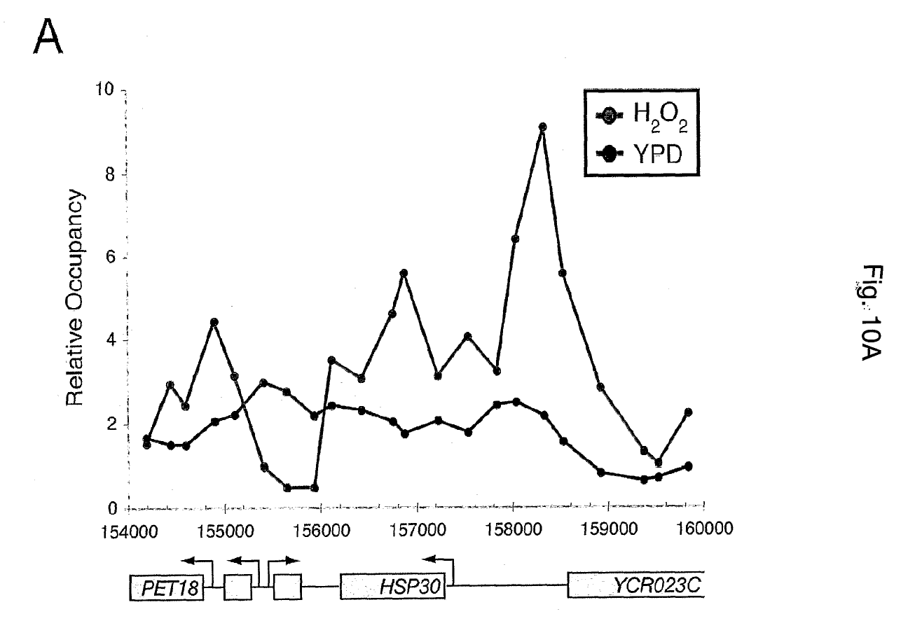

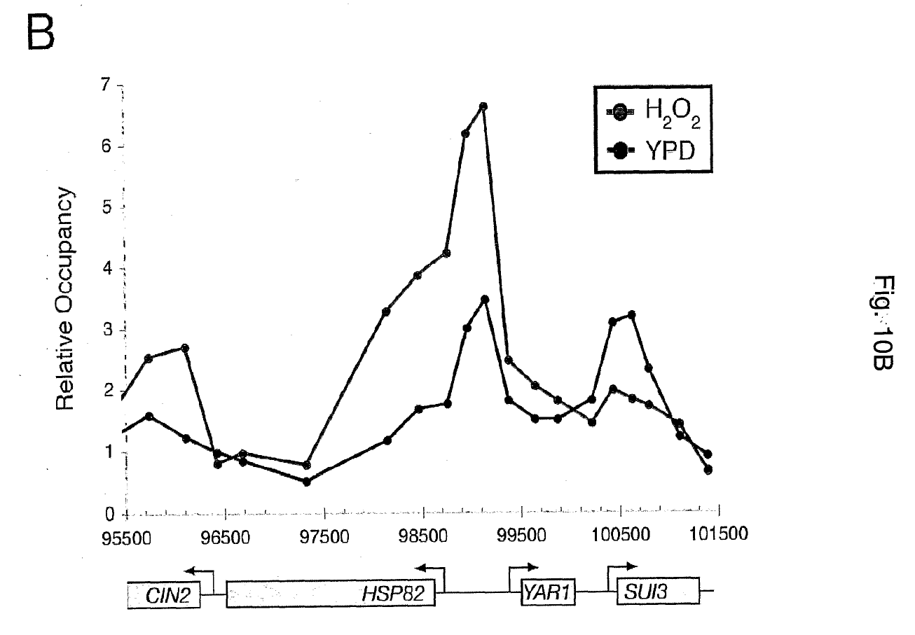

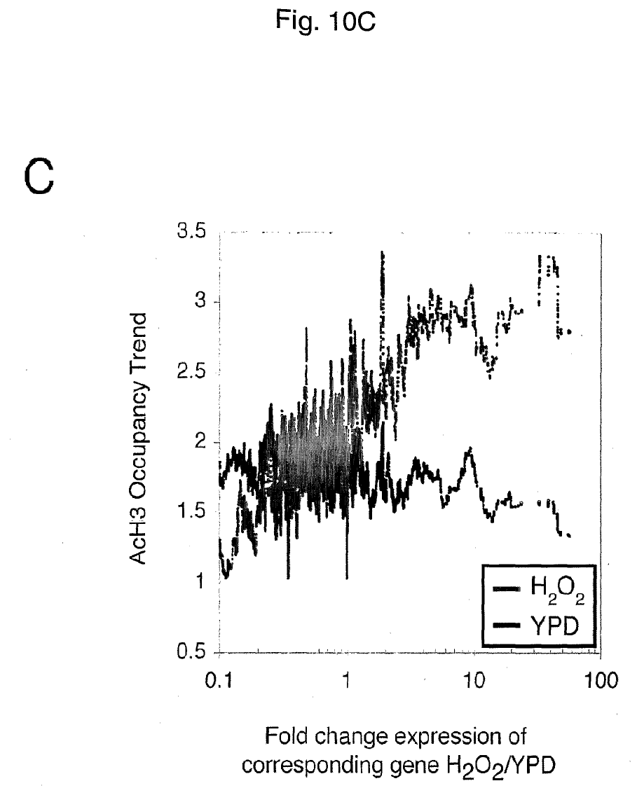

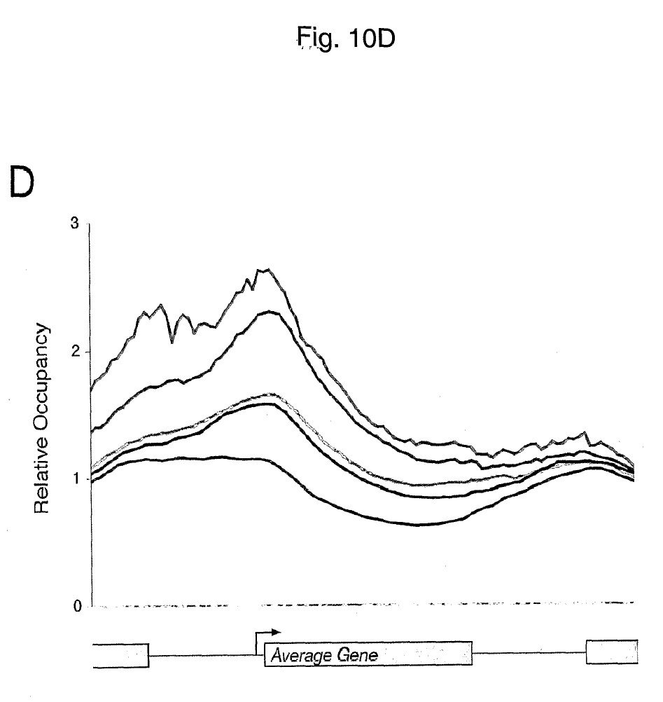

To ascertain whether dynamic gene activation leads to the expected increase in histone acetylation at site catalyzed by Gcn5 and Esa1, Applicants performed ChIP-Chip with the relevant histone antibody on cells before and after exposure to oxidative stress. The results confirm that gene activation leads to increased histone acetylation at sites catalyzed by Gcn5 and Esa1 in the promoter and transcribed regions of activated genes (FIG. 10).

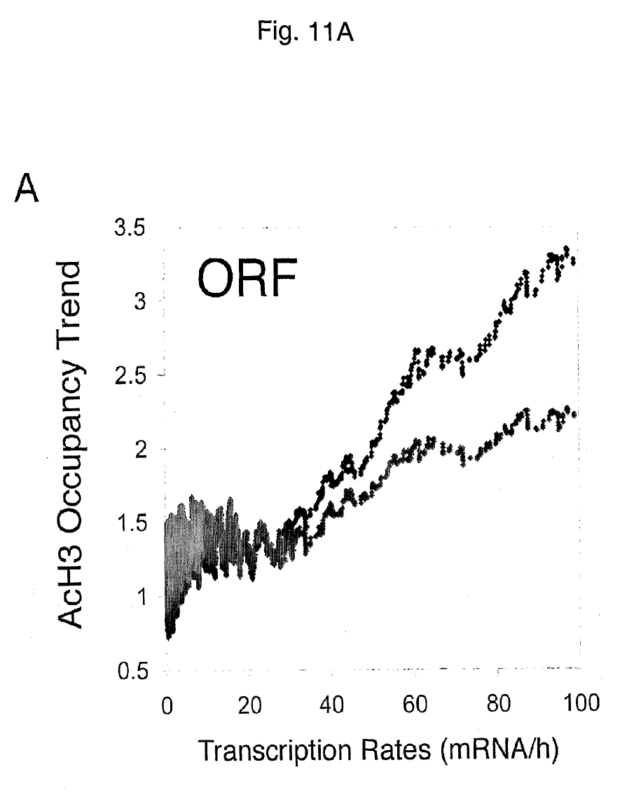

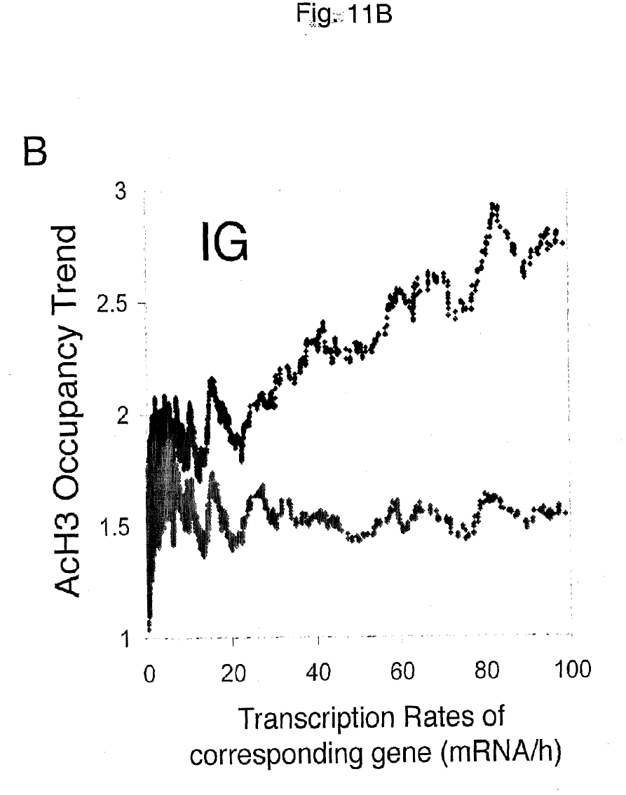

In general, Applicants find that histones with the acetylated residues studied here are enriched predominantly at promoter regions and transcriptional start sites of active genes, and that enrichment drops substantially across the ORFs (FIG. 3, FIG. 10). This is consistent with the model that transcriptional activators generally recruit Gcn5 and Esa1 to promoters of genes upon their activation (Robert et al., 2004) and with the idea that the two HATs acetylate local nucleosomes when recruited to these genes. Our conclusion that there is a strong correlation between transcriptional activity and acetylation of the histone H3 and H4 amino acid residues targeted by Gcn5 and Esa1 is in contrast to that of Kurdistani et al. (2004). This discrepancy is most likely due to differences in the material used in the control channel in the ChIP-Chip procedure. The experiments described here compare ChIP with a histone modification antibody to a control ChIP with a core histone antibody. The experiments reported in Kurdistani et al. (2004) used whole genomic DNA in the reference channel. Applicants found that they could replicate the results in Kurdistani et al. (2004) if Applicants used whole genomic DNA as a reference in ChIP-Chip experiments (FIG. 11), but for reasons described above, this method of normalization is inappropriate.

### Histone Methylation

Methylation of histones in S. cerevisiae is carried out by three known histone methyltransferases, which are capable of covalently modifying specific lysine residues in histone H3 with up to three methyl groups (Peterson and Laniel, 2004). Applicants sought to systematically profile mono-, di- and trimethylated residues at K4, K36 and K79 of histone H3 in nucleosomes associated with genomic DNA.

Applicants measured histone H3K4 trimethylation (H3K4me3) using ChIP-Chip and found that the results and provide a higher resolution picture of H3K4 trimethylation across the yeast genome (FIG. 4A, B). Peaks of histone H3K4 trimethylation occurred at the beginning of actively transcribed genes and there was a positive correlation between this modification and transcription rates (FIG. 4A, B).

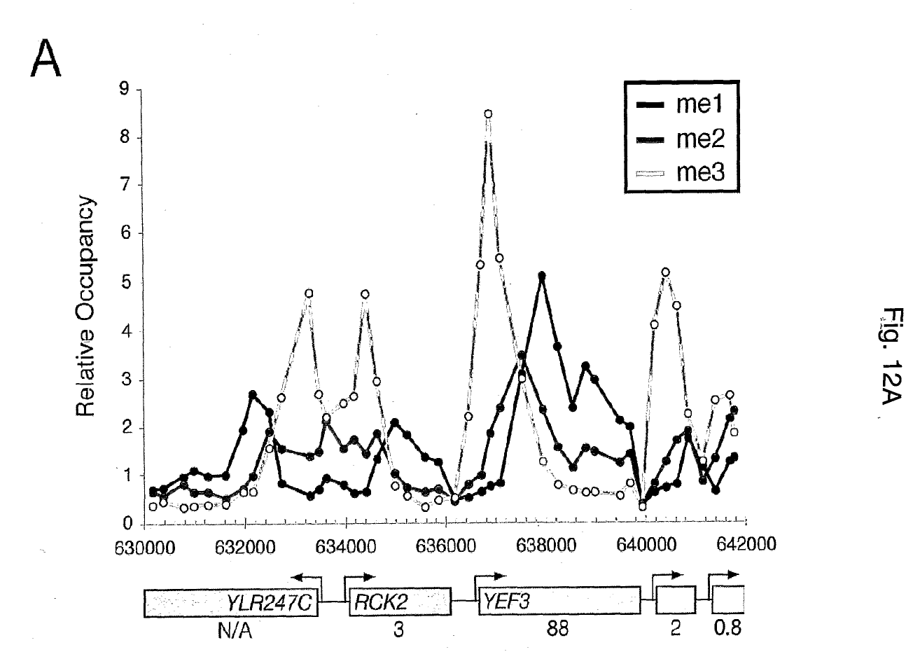

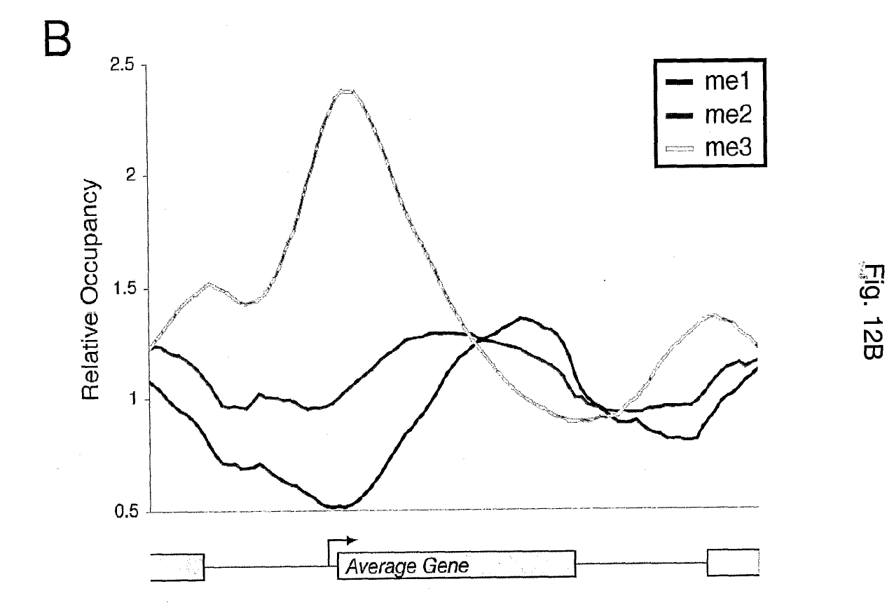

Applicants also investigated the profiles of mono- and dimethylated histone H3K4-containing nucleosomes and found that they exhibit a pattern distinct from that observed for trimethylated histone H3K4 (FIG. 12). While trimethylated H3K4 peaks at the beginning of the transcribed portions of genes, dimethylated H3K4 (H3K4me2) is most enriched in the middle of genes, and monomethylated H3K4 (H3K4me) is found predominantly at the end of genes.

Applicants measured genome-wide the relative levels of H3K36 trimethylation, which is catalyzed by Set2, a factor associated with the later stages of transcriptional elongation (Strahl et al., 2002). In contrast to the pattern observed with H3K4 trimethylated histones, Applicants found that trimethylated H3K36 (H3K36me3) was enriched throughout the coding region, peaking near the 3′ ends of transcription units (FIG. 4C, D). H3K36 trimethylation also correlated with transcriptional activity. These results are consistent with the model that Set2 is recruited by the transcription elongation apparatus and that it methylates local nucleosomes during active transcription.

The Dot1 histone methyltransferase modifies histone H3 lysine 79 (H3K79), which occurs within the core domain of histone H3 (Feng et al., 2002; Ng et al., 2003a; Ng et al., 2002a). Methylation of this residue is estimated to occur in ˜90% of all histones. Applicants investigated the genomic profile of H31(79 trimethylation in yeast (H3K79me3) and found that histones with this modification are enriched within the transcribed regions of genes (FIG. 4E, F). Most genes appeared to have nucleosomes modified at H3K79; there was little correlation between the relative levels of H3K79 trimethylation at genes and transcriptional activity (FIG. 4F).

### Global Map of Histone Marks

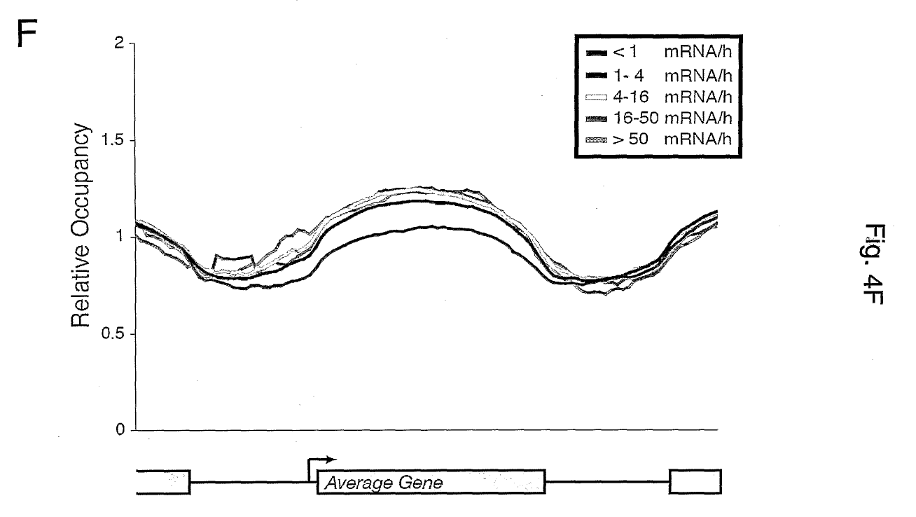

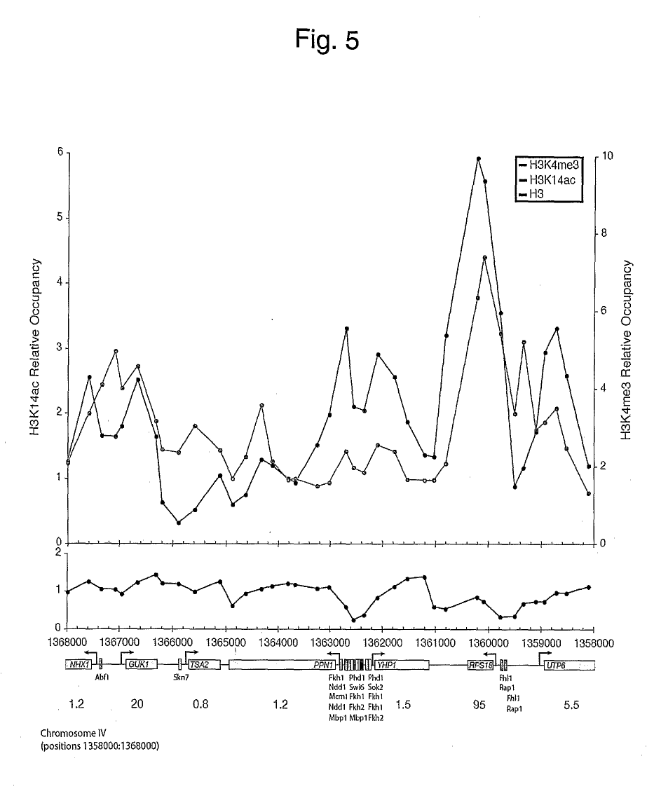

Applicants recently mapped the locations of conserved transcription factor binding sites throughout the yeast genome (Harbison et al., 2004). Applicants used the results described here to generate a complementary genome-wide map of nucleosome occupancy and histone modifications that includes results for eight sets of histone modifications (H3K9ac, H3K14ac, H4K5ac8ac12ac16ac, H3K4me1, H3K4me2, H3K4me3, H3K36me3, and H3K79me3). A portion of this map is shown in FIG. 5 and a browsable form of the complete yeast genome chromatin map is available at the authors' website (http://web.wi.mit.edu/young/nucleosome). A subset of these histone modifications have been examined under two different growth conditions and these results are also available at the authors' website.

### CONCLUDING REMARKS

It is well established that nucleosomes play fundamentally important roles in the organization and maintenance of the genome. Nucleosome modifications have been shown to be associated with transcriptional regulation at well-studied genes and models have emerged that connect regulation of gene expression to histone modification by specific chromatin regulators (Cosma et al., 1999; Gregory et al., 1999; Kuo et al., 1998; Reinke and Horz, 2003). Applicants have carried out a systematic genome-wide analysis of nucleosome acetylation and methylation at sufficient resolution to determine whether models that connect regulation of gene expression to histone modification (Deckert and Struhl, 2001; Reid et al., 2000; Reinke et al., 2001; Suka et al., 2002) apply to gene regulation throughout the yeast genome.

The results described here are consistent with the following general model connecting gene expression to histone modification. Transcriptional activation by DNA-binding regulators generally involves recruitment of Gcn5 and Esa1 to promoters, where these HATs acetylate specific residues on histones H3 and H4 at local nucleosomes (FIG. 3). Applicants were able to find few exceptions to this general rule, where only one or the other HAT acetylates its target residues at the promoters of actively transcribed genes. Active transcription is characteristically accompanied by histone H3K4 trimethylation by Set1 at the beginning of genes (FIG. 4B), and by H3K4 dimethylation and monomethylation at nucleosomes positioned further downstream in the transcription unit (FIG. 12). As the transcription apparatus proceeds down the transcription unit, increasing levels of histone H3K36 trimethylation are observed at most active genes, catalyzed by Set2 (FIG. 4D). Histone H3K79me3, which is catalyzed by Dot1 (Feng et al., 2002; Ng et al., 2003a; van Leeuwen et al., 2002), is enriched within genes, but unlike the other modifications studied here, this enrichment is not clearly associated with active transcription (FIG. 4F). Correlations between transcriptional activity and histone occupancy or modification at intergenic and transcribed regions are summarized in Table 2, as follows:

Table 2 shows the correlation of transcriptional activity and nucleosome occupancy or Modification.

| Specificity | Correlation |
|---|---|
| iH3 | −0.2915071 |
| iH4 | −0.2753623 |
| iH3K9ac | 0.3375921 |
| iH3K14ac | 0.28140397 |
| iH4K5acK8acK12acK16ac | 0.07933304 |
| iH3K4me | −0.0005931 |
| iH3K4me2 | 0.00558487 |
| iH3K4me3 | 0.18052056 |
| iH3K36me3 | 0.32378278 |
| iH3K79me3 | −0.0194656 |
| oH3 | −0.2304769 |
| oH4 | −0.2621408 |
| oH3K9ac | 0.29472382 |
| oH3K14ac | 0.37440913 |
| oH4K5acK8acK12acK16ac | 0.10454317 |
| oH3K4me | −0.0657816 |
| oH3K4me2 | 0.09084053 |
| oH3K4me3 | 0.40914098 |
| oH3K36me3 | 0.40710237 |
| oH3K79me3 | 0.0474006 |

In the table above, the prefix “i” denotes correlation with intergenic probes; the prefix “o” denotes correlation with ORF probes; The correlation refers to the correlation coefficient between ratio enrichment and transcriptional activity in mRNA/hr (Holstege et al.).

The genome-wide maps of histone occupancy and modification described here should provide investigators with information useful for further exploring the histone code and its implications for gene regulation and chromosome organization and maintenance. Applicants expect that the approaches used here to map histone occupancy and modification in yeast can also be used to gain insights into the linkage between gene expression and histone modification across the genome in higher eukaryotes.

### Experimental Procedures

### Array Design

The Agilent DNA microarray used here has 44,290 features consisting of 60-mer oligonucleotide probes. The array covers 12 Mb of the yeast genome (85%), excluding highly repetitive regions, with an average probe density of 266 bp. Intergenic regions are represented by 14,256 probes and ORFs are represented by 27,185 probes. The remaining 2,849 control features included blank spots and probes derived from Arabidopsis thaliana.

### Epitope Tagging, Antibodies and Strains

Transcriptional and chromatin regulators were tagged at the C-terminus with a 9-copy myc epitope. The sequence encoding the myc epitope was introduced into the endogenous gene immediately upstream of the stop codon. Specific oligonucleotides were used to generate PCR products from plasmids described by Cosma et al. (Cosma et al., 1999). The resulting PCR products were transformed into a W303 yeast to generate the tagged strains by one-step genomic integration. Clones were selected for growth on the appropriate selective media plates, and the insertion was confirmed by PCR. The expression of the epitope-tagged protein was confirmed by western blotting using an anti-Myc (9E11). The antibodies used in this study are listed as follows:

| Specificity | Supplier | Catalog # |
|---|---|---|
| Anti-Histone H3 | Abeam | ab1791 |
| Anti-Histone H4 | Abeam | ab10156 |
| Anti-H3K9ac | Upstate Biotechnology | 06-942 |
| Anti-H3K14ac | Upstate Biotechnology | 06-911 |
| Anti-H4ac | Upstate Biotechnology | 06-866 |
| Anti-H3K4me3 | Abeam | ab8580 |
| Anti-H3K4me2 | Abeam | ab7766 |
| Anti-H3K4me1 | Abcam | ab8895 |
| Anti-H3K36me3 | Abcam | ab9050 |
| Anti-H3K79me3 | Abeam | ab2621 |
| Anti-myc | Abcam | 9E11 |
| Rabbit IgG | Upstate Biotechnology | 12-370 |

### Chromatin Immunoprecipitation and Genome-Wide ChIP-Chip

Chromatin immunoprecipitation and genome-wide location analysis were performed as described previously (Ren et al., 2000) except that the crosslinking time was reduced to 30 minutes at room temperature, the order of proteinase K and RNase treatment was reversed, and high resolution oligonucleotide arrays (Agilent Technologies) were used for hybridizations. A detailed protocol can be found at http://web.wi.mit.edu/young/nucleosome. Briefly, yeast cells were grown in at least in two independent cultures in rich medium. Response to hydrogen peroxide was induced by adding hydrogen peroxide to the cell cultures grown at mid-log phase in YPD medium at 30° C. to final concentration of 0.4 mM for 20 minutes. Cultures were treated with formaldehyde (1%) for 30 minutes, cells were collected by centrifugation, washed with ice-cold TBS and disrupted by vortexing in lysis buffer in the presence of glass beads. The chromatin was sonicated to yield an average DNA fragment of 500 bps. The DNA fragments crosslinked to the proteins were enriched by immunoprecipitation with specific antibodies. After reversal of the crosslinks and purification the immunoprecipitated and input DNA was labeled by ligation-mediated PCR with Cy5 and Cy3 fluorescent dyes, respectively. Both pools of labeled DNA were hybridized to a single DNA microarray (described above). Images of Cy5 and Cy3 fluorescence intensities were generated by scanning array using GenePix 5000 scanner and were analyzed with GenePix Pro 5.1 software. Experiments were carried out at least in duplicate. All microarray data are available from ArrayExpress (E-WMIT-3) and from the authors' website.

Applicants and other groups have noted that there can be modest differences in the relative levels of intergenic and genic yeast DNA that are recovered during phenol extraction (Nagy et al., 2003). Experimental analysis indicates that this is not due to differences in our ability to detect intergenic and genic DNA on the DNA microarrays. Our experiments also indicate that this observation is not due to artifacts due to differential labeling of DNA. Others have speculated that differential recovery is due to contaminating nucleases that might preferentially digest intergenic DNA (Nagy et al., 2003). It is also possible that there are intrinsic differences in susceptibility to shearing by sonication in intergenic and genic DNA.

### Mock Immunoprecipitation Normalization

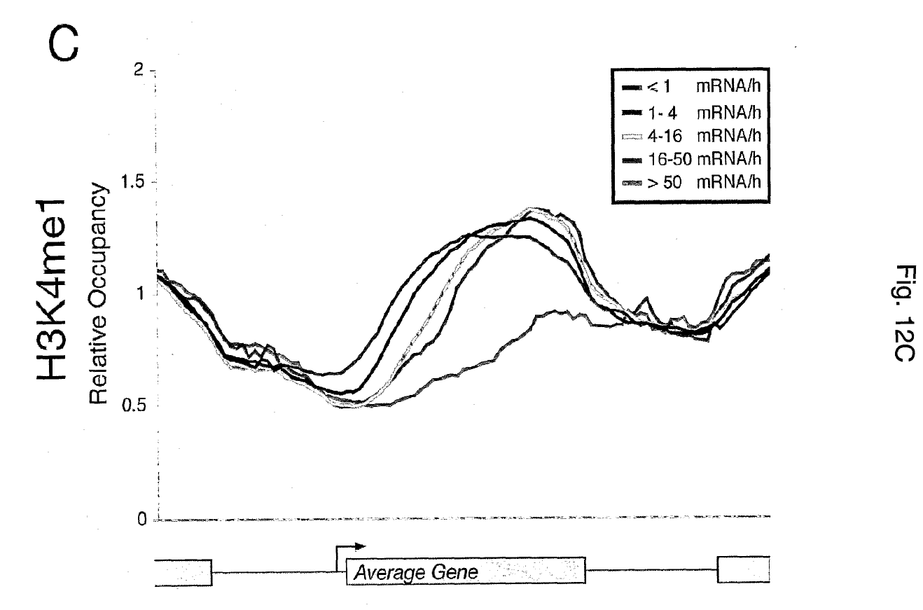

Control immunoprecipitations were performed as above with two exceptions. In one case, an antibody with no specificity to histones (Rabbit IgG) was substituted for the H3- or H4-specific antibody. In the second case, no antibody was added during the overnight incubation with magnetic beads. For histone H3 and H4 (not shown), data were normalized relative to the “no antibody” control. Distributions of relative occupancy at ORF and intergenic regions by histone H3 are depicted in FIG. 13. Following normalization by this method the standard deviation is 0.38, and Applicants used two-sampled T-tests to determine the likelihood of differences occurring by chance in both the original and controlled experiments (FIG. 13).

### Data Analysis

Genome-wide location data were subjected to quality control filters, median-normalized, and the weighted average ratio of immunoprecipitated to control DNA was determined for each spot across all replicates. A confidence value (P value) for single probes and an averaged confidence value for neighboring probes were calculated. A detailed description of the error model is available at the authors' website.

A binding cutoff for Gcn4 was determined by comparing maximum IP/WCE ratios to high likelihood positive and high likelihood negative list using ROC curve analysis. A positive list of 84 genes (Table 1) was selected on the basis of previous high confidence binding data (P≦0.001) (Harbison et al., 2004), the presence of a perfect or near perfect Gcn4 consensus binding site (TGASTCA) in the region of −400 bp to +50 bp, and a greater then 2-fold change in steady state mRNA levels dependent on Gcn4 when shifted to amino acid starvation medium (Natarajan et al., 2001). The negative list of 945 genes not transcribed from divergent intergenic regions was selected by weak binding (P≧0.1), absence of a motif near the presumed start site, and less then a 60% change in steady state mRNA levels in response to shift to amino acid starvation. Each gene was scored based on the minimum P value found in the region −250 to +50 bp from the UAS using the higher of the single and averaged confidence score. Optimal parameters were determined by maximizing the absolute difference in identified genes in both the positive list and negative lists using the Statistics-ROC package for Perl.

Nucleosome-depleted promoters were defined as intergenic regions upstream of protein-coding genes for which unmodified histone H3 or H4 enrichment met the following criterion: the enrichment of any probe within the intergenic region was less than the average ratio of enrichment at two neighboring ORFs.

Throughout the text histone H3 occupancy is often referred to as nucleosome occupancy. There are two reasons to believe that the results with H3 likely reflect nucleosome occupancy and not nucleosomes that are missing H3 specifically. First, Applicants obtain similar results in independent experiments with 1H3 and H4. Second, previous in vitro studies suggest that it is H2A-H₂B dimers (and not H3 or H4) that preferentially dissociate from nucleosomes during transcription (Kireeva et al., 2002).

### REFERENCES

Allard, S., Utley, R. T., Savard, J., Clarke, A., Grant, P., Brandl, C. J., Pillus, L., Workman, J. L., and Cote, J. (1999). NuA4, an essential transcription adaptor/histone H4 acetyltransferase complex containing Esa1p and the ATM-related cofactor Tralp. Embo J 18, 5108-5119.
Arndt, K., and Fink, G. R. (1986). GCN4 protein, a positive transcription factor in yeast, binds general control promoters at all 5′ TGACTC 3′ sequences. Proc Natl Acad Sci USA 83, 8516-8520.
Bannister, A. J., Schneider, R., Myers, F. A., Thorne, A. W., Crane-Robinson, C., and Kouzarides, T. (2005). Spatial distribution of di- and tri-methyl lysine 36 of histone H3 at active genes. J Biol. Chem.
Bernstein, B. E., Humphrey, E. L., Erlich, R. L., Schneider, R., Bouman, P., Liu, J. S., Kouzarides, T., and Schreiber, S. L. (2002). Methylation of histone H3 Lys 4 in coding regions of active genes. Proc Natl Acad Sci USA 99, 8695-8700.
Bernstein, B. E., Kamal, M., Lindblad-Toh, K., Bekiranov, S., Bailey, D. K., Huebert, D. J., McMahon, S., Karlsson, E. K., Kulbokas, E. J., 3rd, Gingeras, T. R., et al. (2005). Genomic maps and comparative analysis of histone modifications in human and mouse. Cell 120, 169-181.
Bernstein, B. E., Liu, C. L., Humphrey, E. L., Perlstein, E. O., and Schreiber, S. L. (2004). Global nucleosome occupancy in yeast. Genome Biol 5, R62.
Bhaumik, S. R., and Green, M. R. (2001). SAGA is an essential in vivo target of the yeast acidic activator Gal4p. Genes Dev 15, 1935-1945.
Boeger, H., Griesenbeck, J., Strattan, J. S., and Kornberg, R. D. (2003). Nucleosomes unfold completely at a transcriptionally active promoter. Mol Cell 11, 1587-1598.
Briggs, S. D., Bryk, M., Strahl, B. D., Cheung, W. L., Davie, J. K., Dent, S. Y., Winston, F., and Allis, C. D. (2001). Histone H3 lysine 4 methylation is mediated by Set1 and required for cell growth and rDNA silencing in Saccharomyces cerevisiae. Genes Dev 15, 3286-3295.
Causton, H. C., Ren, B., Koh, S. S., Harbison, C. T., Kanin, E., Jennings, E. G., Lee, T. I., True, H. L., Lander, E. S., and Young, R. A. (2001). Remodeling of yeast genome expression in response to environmental changes. Mol Biol Cell 12, 323-337.
Clarke, A. S., Lowell, J. E., Jacobson, S. J., and Pillus, L. (1999). Esa1p is an essential histone acetyltransferase required for cell cycle progression. Mol Cell Biol 19, 2515-2526.
Cosma, M. P., Tanaka, T., and Nasmyth, K. (1999). Ordered recruitment of transcription and chromatin remodeling factors to a cell cycle- and developmentally regulated promoter. Cell 97, 299-311.
Deckert, J., and Struhli, K. (2001). Histone acetylation at promoters is differentially affected by specific activators and repressors. Mol Cell Biol 21, 2726-2735.
Ellenberger, T. E., Brandl, C. J., Struhl, K., and Harrison, S.C. (1992). The GCN4 basic region leucine zipper binds DNA as a dimer of uninterrupted alpha helices: crystal structure of the protein-DNA complex. Cell 71, 1223-1237.
Feng, Q., Wang, H., Ng, H. H., Erdjument-Bromage, H., Tempst, P., Struhl, K., and Zhang, Y. (2002). Methylation of H3-lysine 79 is mediated by a new family of HMTases without a SET domain. Curr Biol 12, 1052-1058.
Gregory, P. D., Schmid, A., Zavari, M., Munsterkotter, M., and Horz, W. (1999). Chromatin remodelling at the PHO8 promoter requires SWI-SNF and SAGA at a step subsequent to activator binding. Embo J 18, 6407-6414.
Harbison, C. T., Gordon, D. B., Lee, T. I., Rinaldi, N. J., Macisaac, K. D., Danford, T. W., Hannett, N. M., Tagne, J. B., Reynolds, D. B., Yoo, J., et al. (2004). Transcriptional regulatory code of a eukaryotic genome. Nature 431, 99-104.
Holstege, F. C., Jennings, E. G., Wyrick, J. J., Lee, T. I., Hengartner, C. J., Green, M. R., Golub, T. R., Lander, E. S., and Young, R. A. (1998). Dissecting the regulatory circuitry of a eukaryotic genolne. Cell 95, 717-728.
Hope, I. A., and Struhl, K. (1985). GCN4 protein, synthesized in vitro, binds HIS3 regulatory sequences: implications for general control of amino acid biosynthetic genes in yeast. Cell 43, 177-188.
Humphrey, E. L., Shamji, A. F., Bernstein, B. E., and Schreiber, S. L. (2004). Rpd3p relocation mediates a transcriptional response to rapamycin in yeast. Chem Biol 11, 295-299.
Kireeva, M. L., Walter, W., Tchernajenko, V., Bondarenko, V., Kashlev, M., Studitsky, V. M., (2002). Nucleosome remodeling induced by RNA polymerase II: loss of the H2A/H₂B dimer during transcription. Mol Cell 9, 541-552.
Kouzarides, T. (2002). Histone methylation in transcriptional control. Cunr Opin Genet Dev 12, 198-209.
Krogan, N. J., Dover, J., Wood, A., Schneider, J., Heidt, J., Boateng, M. A., Dean, K., Ryan, O. W., Golshani, A., Johnston, M., et al. (2003a). The Paf1 complex is required for histone H3 methylation by COMPASS and Dot1p: linking transcriptional elongation to histone methylation. Mol Cell 11, 721-729.
Krogan, N. J., Kim, M., Tong, A., Golshani, A., Cagney, G., Canadien, V., Richards, D. P., Beattie, B. K., Emili, A., Boone, C., et al. (2003b). Methylation of histone H3 by Set2 in Saccharomyces cerevisiae is linked to transcriptional elongation by RNA polymerase II. Mol Cell Biol 23, 4207-4218.
Kuo, M. H., Brownell, J. E., Sobel, R. E., Ranalli, T. A., Cook, R. G., Edmondson, D. G., Roth, S. Y., and Allis, C. D. (1996). Transcription-linked acetylation by Gcn5p of histones H3 and H4 at specific lysines. Nature 383, 269-272.
Kuo, M. H., Zhou, J., Jambeck, P., Churchill, M. E., and Allis, C. D. (1998). Histone acetyltransferase activity of yeast Gcn5p is required for the activation of target genes in vivo. Genes Dev 12, 627-639.
Kurdistani, S. K., Robyr, D., Tavazoie, S., and Grunstein, M. (2002). Genome-wide binding map of the histone deacetylase Rpd3 in yeast. Nat Genet. 31, 248-254.
Kurdistani, S. K., Tavazoie, S., and Grunstein, M. (2004). Mapping global histone acetylation patterns to gene expression. Cell 117, 721-733.
Larschan, E., and Winston, F. (2001). The S. cerevisiae SAGA complex functions in vivo as a coactivator for transcriptional activation by Gal4. Genes Dev 15, 1946-1956.
Lee, C. K., Shibata, Y., Rao, B., Strahl, B. D., and Lieb, J. D. (2004). Evidence for nucleosome depletion at active regulatory regions genome-wide. Nat Genet. 36, 900-905.
Lee, T. I., Rinaldi, N.J., Robert, F., Odom, D. T., Bar-Joseph, Z., Gerber, G. K., Hannett, N. M., Harbison, C. T., Thompson, C. M., Simon, I., et al. (2002). Transcriptional regulatory networks in Saccharomyces cerevisiae. Science 298, 799-804.
Lieb, J. D., Liu, X., Botstein, D., and Brown, P. O. (2001). Promoter-specific binding of Rap1 revealed by genome-wide maps of protein-DNA association. Nat Genet. 28, 327-334.
Luger, K., Mader, A. W., Richmond, R. K., Sargent, D. F., and Richmond, T. J. (1997). Crystal structure of the nucleosome core particle at 2.8 A resolution. Nature 389, 251-260.
Nagy, P. L., Cleary, M. L., Brown, P. O., and Lieb, J. D. (2003). Genomewide demarcation of RNA polymerase II transcription units revealed by physical fractionation of chromatin. Proc Natl Acad Sci USA 100, 6364-6369.
Narlikar, G. J., Fan, H. Y., and Kingston, R. E. (2002). Cooperation between complexes that regulate chromatin structure and transcription. Cell 108, 475-487.
Natarajan, K., Meyer, M. R., Jackson, B. M., Slade, D., Roberts, C., Hinnebusch, A. G., and Marton, M. J. (2001). Transcriptional profiling shows that Gcn4p is a master regulator of gene expression during amino acid starvation in yeast. Mol Cell Biol 21, 4347-4368.
Ng, H. H., Ciccone, D. N., Morshead, K. B., Oettinger, M. A., and Struhl, K. (2003a). Lysine-79 of histone H3 is hypomethylated at silenced loci in yeast and mammalian cells: a potential mechanism for position-effect variegation. Proc Natl Acad Sci USA 100, 1820-1825.
Ng, H. H., Feng, Q., Wang, H., Erdjument-Bromage, H., Tempst, P., Zhang, Y., and Struhl, K. (2002a). Lysine methylation within the globular domain of histone H3 by Dot1 is important for telomeric silencing and Sir protein association. Genes Dev 16, 1518-1527.
Ng, H. H., Robert, F., Young, R. A., and Struhl, K. (2002b). Genome-wide location and regulated recruitment of the RSC nucleosome-remodeling complex. Genes Dev 16, 806-819.
Ng, H. H., Robert, F., Young, R. A., and Struhl, K. (2003b). Targeted recruitment of Set1 histone methylase by elongating Pol II provides a localized mark and memory of recent transcriptional activity. Mol Cell 11, 709-719.
O'Shea, E. K., Klemm, J. D., Kim, P. S., and Alber, T. (1991). X-ray structure of the GCN4 leucine zipper, a two-stranded, parallel coiled coil. Science 254, 539-544.
Oliphant, A. R., Brandl, C. J., and Struhl, K. (1989). Defining the sequence specificity of DNA-binding proteins by selecting binding sites from random-sequence oligonucleotides: analysis of yeast GCN4 protein. Mol Cell Biol 9, 2944-2949.
Peterson, C. L., and Laniel, M. A. (2004). Histones and histone modifications. Curr Biol 14, R546-551.
Reid, J. L., Iyer, V. R., Brown, P. O., and Struhl, K. (2000). Coordinate regulation of yeast ribosomal protein genes is associated with targeted recruitment of Esa1 histone acetylase. Mol Cell 6, 1297-1307.
Reinke, H., Gregory, P. D., and Horz, W. (2001). A transient histone hyperacetylation signal marks nucleosomes for remodeling at the PHO8 promoter in vivo. Mol Cell 7, 529-538.
Reinke, H., and Horz, W. (2003). Histones are first hyperacetylated and then lose contact with the activated PHO5 promoter. Mol Cell 11, 1599-1607.
Ren, B., Robert, F., Wyrick, J. J., Aparicio, O., Jennings, E. G., Simon, I., Zeitlinger, J., Schreiber, J., Hannett, N., Kanin, E., et al. (2000). Genome-wide location and function of DNA-binding proteins. Science 290, 2306-2309.
Robert, F., Pokholok, D. K., Hannett, N. M., Rinaldi, N. J., Chandy, M., Rolfe, A., Workman, J. L., Gifford, D. K., and Young, R. A. (2004). Global Position and Recruitment of HATs and HDACs in the Yeast Genome. Mol Cell 16, 199-209.
Robyr, D., Suka, Y., Xenarios, I., Kurdistani, S. K., Wang, A., Suka, N., and Grunstein, M. (2002). Microarray deacetylation maps determine genome-wide functions for yeast histone deacetylases. Cell 109, 437-446.
Roh, T. Y., Ngau, W. C., Cui, K., Landsman, D., and Zhao, K. (2004). High-resolution genome-wide mapping of histone modifications. Nat Biotechnol 22, 1013-1016.
Santos-Rosa, H., Sclmeider, R., Bannister, A. J., Sherriff, J., Bernstein, B. E., Emre, N.C., Schreiber, S. L., Mellor, J., and Kouzarides, T. (2002). Active genes are tri-methylated at K4 of histone H3. Nature 419, 407-411.
Schubeler, D., MacAlpine, D. M., Scalzo, D., Wirbelauer, C., Kooperberg, C., van Leeuwen, F., Gottschling, D. E., O'Neill, L. P., Turner, B. M., Delrow, J., et al. (2004). The histone modification pattern of active genes revealed through genome-wide chromatin analysis of a higher eukaryote. Genes Dev 18, 1263-1271.
Strahl, B. D., Grant, P. A., Briggs, S. D., Sun, Z. W., Bone, J. R., Caldwell, J. A., Mollah, S., Cook, R. G., Shabanowitz, J., Hunt, D. F., and Allis, C. D. (2002). Set2 is a nucleosomal histone H3-selective methyltransferase that mediates transcriptional repression. Mol Cell Biol 22, 1298-1306.
Suka, N., Luo, K., and Grunstein, M. (2002). Sir2p and Sas2p opposingly regulate acetylation of yeast histone H4 lysine16 and spreading of heterochromatin. Nat Genet. 32, 378-383.
Utley, R. T., Ikeda, K., Grant, P. A., Cote, J., Steger, D. J., Eberharter, A., John, S., and Workman, J. L. (1998). Transcriptional activators direct histone acetyltransferase complexes to nucleosomes. Nature 394, 498-502.
van Leeuwen, F., Gafken, P. R., and Gottschling, D. E. (2002). Dot1p modulates silencing in yeast by methylation of the nucleosome core. Cell 109, 745-756.
Vogelauer, M., Wu, J., Suka, N., and Grunstein, M. (2000). Global histone acetylation and deacetylation in yeast. Nature 408, 495-498.
Xiao, T., Hall, H., Kizer, K. O., Shibata, Y., Hall, M. C., Borchers, C. H., and Strahl, B. D. (2003). Phosphorylation of RNA polymerase II CTD regulates H3 methylation in yeast. Genes Dev 17, 654-663.
Zhang, W., Bone, J. R., Edmondson, D. G., Turner, B. M., and Roth, S. Y. (1998). Essential and redundant functions of histone acetylation revealed by mutation of target lysines and loss of the Gcn5p acetyltransferase. Embo J 17, 3155-3167.

We claim:

1. A method for identifying regions of a genome to which a protein of interest binds, the method comprising the steps of:
(a) producing a mixture comprising DNA fragments to which the protein of interest is bound;
(b) isolating one or more DNA fragments to which the protein of interest is bound from the mixture produced in step (a); and
(c) generating probes from the one or more of the isolated DNA fragments;
(d) identifying one or more regions of the genome which are complementary to the probe fragments isolated in step (c) by combining the probe with a tiled array comprising one or more sets of distinct oligonucleotide features bound to a surface of a solid support, wherein the distinct oligonucleotide features are each complementary to a region of the genome,
thereby identifying regions of the genome to which the protein of interest binds.

2. The method of claim 1, wherein step (d) comprises combining the probe and the tiled array under conditions in which specific hybridization between the probe and the oligonucleotide features can occur, and detecting said hybridization, wherein hybridization between the labeled probe and a oligonucleotide feature relative to a suitable control indicates that the protein of interest is bound to the region of the genome to which the sequence of the oligonucleotide feature is complementary.

3. The method of claim 1, wherein the probe is labeled with a fluorescent probe.

4. The method of claim 1, wherein one or more sets of the distinct oligonucleotide features are complementary to locations in the genome that are substantially evenly spaced.

5. The method of claim 5, wherein the distinct oligonucleotide features are complementary to adjacent regions in the genome are spaced from 10 bp to 20 kb of each other.

6. The method of claim 5, wherein the distinct oligonucleotide features are complementary to adjacent regions in the genome are spaced from 20 bp to 10 kb of each other.

7. The method of claim 1, wherein the oligonucleotide features comprise DNA or RNA or modified forms thereof.

8. The method of claim 7 wherein the modified forms of DNA are PNA or LNA molecules.

9. The method according to claim 1, wherein said oligonucleotide features comprise nucleic acids that range in size from about 20 nt to about 200 nt in length.

10. The method according to claim 9, wherein said nucleic acids range in size from about 20 to about 100 nt in length.

11. The method according to claim 10, wherein said nucleic acids range in size from about 40 to about 80 nt in length.

12. The method according to claim 1, wherein said oligonucleotide features bound to a surface of a solid support includes sequences representative of locations distributed across at least a portion of a genome.

13. The method according to claim 12, wherein said locations have a uniform spacing across at least a portion of a genome.

14. The method according to claim 12, wherein said locations have a non-uniform spacing across at least a portion of a genome.

15. The method according to claim 1, wherein the one or more sets of oligonucleotide features bound to a surface of a solid support samples the portion of the genome at least about every 20 Kb.

16. The method according to claim 1, wherein the one or more sets of oligonucleotide features bound to a surface of a solid support samples at least a portion of the genome at least about every 2 Kb.

17. The method according to claim 1, wherein the one or more sets of oligonucleotide features bound to a surface of a solid support samples at least a portion of the genome at least about every 0.5 Kb.

18. The method of claim 12, wherein the portion of the genome comprises at least 20% of the genome.

19. The method of claim 12, wherein the portion of the genome comprises regions of at least at least 20% of chromosomes in the genome.

20. The method according to claim 1, wherein at least one set of distinct oligonucleotide features comprises distinct oligonucleotide features that correspond to non-coding genomic regions.

21. The method according to claim 20, wherein at least 50% of said sets of distinct oligonucleotide features are complementary to non-promoter regions.

22. The method according to claim 1, wherein at least one set of distinct oligonucleotide features comprises distinct oligonucleotide features that correspond to coding genomic regions.

23. The method according to claim 21, wherein at least 50% of the distinct oligonucleotide features that correspond to coding genomic regions do not comprise entire open reading frames.

24. The method according to claim 1, wherein the solid support is a planar substrate.

25. The method according to claim 24, wherein said planar substrate is glass.

26. The method of claim 1, wherein the protein of interest is a histone.

27. The method of claim 53, wherein the histone is a modified histone.

28. The method of claim 55, wherein the histone is acetylated, methylated, phosphorylated, or combinations thereof.

29. The method of claim 55, wherein the histone is H3 or H4.

30. The method of claim 1, wherein steps (a), (b) and/or (c) are performed in a first location, and step (d) is performed in a second location, wherein the first location is remote to the second location.

31. The method of claim 30, further comprising a data transmission step between the first location and the second location.

32. The method of claim 31, wherein the data transmission step occurs via an electronic communication link.

33. The method of claim 32, wherein the data communication link is the internet.

34. The method of claim 33, wherein the data transmission step from the first to the second location comprises experimental parameter data, wherein the experimental parameter data comprises data selected from:
(a) the phylogenetic species of the genome;
(b) clinical data from the organism from which the genome was derived; and
(c) a microarray to which the labeled probes are to be hybridized.

35. The method of claim 34, wherein the data transmission step from the second location to the first location comprises (i) one or more data transmission substeps from the second location to one or more intermediate location; and (b) one or more data transmission substeps from one or more intermediate location to the first location, wherein the intermediate locations are remote to both the first and second locations.

36. The method of claim 29, further comprising a data transmission step in which a result from identifying regions of a genome is transmitted from the second location to the first location.

37. The method of claim 36, wherein the data transmission step from the second location to the first location comprises (i) one or more data transmission substeps from the second location to one or more intermediate location; and (b) one or more data transmission substeps from one or more intermediate location to the first location, wherein the intermediate locations are remote to both the first and second locations.

38. The method of claim 36, wherein the data transmission step occurs via the an electronic communication link.

39. The method of claim 38, wherein the data communication link is the internet.

40. The method of claim 1, wherein the genome is from an eukaryotic cell.

41. The method of claim 40, wherein the cell is a metazoan cell.

42. The method of claim 41, wherein the cell is a mammalian cell.

43. The method of claim 40, wherein the cell is a primary cell.

44. The method of claim 43, wherein the cell is derived from a tissue biopsy.

45. The method of claim 44, wherein the tissue biopsy is from a subject afflicted with, or suspected of being afflicted with, a disorder.

46. The method of claim 42, wherein the cell is a human cell.

47. The method of claim 40, wherein the cell is a yeast cell.

48. The method of claim 1, wherein the protein of interest is a sequence-specific DNA-binding protein.

49. The method of claim 1, wherein the protein of interest is not a sequence-specific DNA-binding protein.

50. The method of claim 1, wherein the protein of interest is acetylated, methylated, or both.

51. The method of claim 1, wherein the protein of interest is native to the cell.

52. The method of claim 1, wherein the protein of interest is a recombinant protein.

53. The method of claim 4, wherein the DNA fragments to which the protein of interest is bound from the mixture produced in step (a), or the labeled probes derived from said DNA fragments, are delivered from the first location to the second location.

54. The method of claim 29, wherein the histone is selected from H3, H4, H3K9ac, H3K14ac, H4K5acK8acK12acK16ac, H3K4me, H3K4me2, H3K4me3, H3K36me3 and H3K79me3.

55. The method of claim 54, wherein the histone is selected from H3K9ac, H3K14ac, H4K5acK8acK12acK16ac, H3K4me, H3K4me2, H3K4me3, H3K36me3 and H3K79me3.

56. The method of claim 54, wherein the histone is selected from H3K9ac, H3K14ac, H4K5acK8acK12acK16ac.

57. The method of claim 55, wherein the histone is selected from H3K4me, H3K4me2, H3K4me3, H3K36me3 and H3K79me3.

58. The method of claim 1, wherein the genome is from a first cell and the protein of interest from a second cell.

59. The method of claim 58, comprising the step, prior to step (a), of contacting the protein of interest with the genome.

60. The method of claim 58, wherein the protein of interest is contacted with the genome ex vivo by contacting
(i) an extract comprising the protein; and
(ii) an extract comprising the genome.

61. The method of claim 58, wherein the protein of interest is a recombinant protein.

62. The method of claim 58, wherein the protein of interest is a naturally-occurring protein.

63. The method of claim 58, wherein the first cell and the second cells are from different species.

64. A method of estimating the transcriptional rate of a gene, the method comprising determining the level of acetylated histone bound to a transcriptional start site of the gene, wherein increased levels of bound acetylated histone indicate a higher transcriptional rate.

65. The method of claim 64, wherein the acetylated histone in monoacetylated.

66. The method of claim 64, wherein the acetylated histone is multiply acetylated.

67. The method of claim 64, wherein determining the relative level of acetylated histone bound to a transcriptional start site of the gene comprises determining the regions of the genome to which the acetylated histone binds using the method of claim 1.

68. The method of claim 64, wherein the acetylated histone is H3 acetylated at K9, H3 acetylated at K14, or H4 acetylated at K5, K8, K12 and K16.

69. A method of estimating the transcriptional rate of a gene, the method comprising determining the level of methylated histone bound to the transcribed region the gene, wherein increased levels of methylated histone bound to the transcribed region indicate a higher transcriptional rate.

70. The method of claim 69, wherein the methylated histone in trimethylated.

71. The method of claim 69, wherein the methylated histone is H3 methylated at K36.

72. The method of claim 69, wherein the methylated histone is H3 trimethylated at K36.

73. The method of claim 69, wherein determining the relative level of methylated histone bound to the transcribed region of a gene comprises determining the regions of the genome to which the methylated histone binds using the method of claim 1.

74. The method of claim 69, wherein the transcribed region or the gene is the coding sequence.
